SEQUENCE LISTING (11 sequences)

SEQ ID NO: 1 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 2 - protein, 208 aa, Homo sapiens [sequence omitted]

SEQ ID NO: 3 - protein, 126 aa, Homo sapiens
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGAQIYVIDPEPCPDSD

SEQ ID NO: 4 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 5 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 607 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPIN

SEQ ID NO: 8 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 55 aa, Homo sapiens
GTSHLVKCAEKEKTFCVNGGECFMVKDLSNPSRYLCKCPNEFTGDRCQNYVMASF

SEQ ID NO: 10 - protein, 9 aa, Homo sapiens
TYLPANASL

SEQ ID NO: 11 - protein, 27 aa, Homo sapiens
NGGTSLSEKTVLLLVTPFLAAAWSLHP

(19) United States
(12) Patent Application Publication
(10) Pub. No.: US 2018/0128833 A1
(43) Pub. Date: May 10, 2018

(54) METHODS OF TREATING WITH TUMOR MEMBRANE VESICLE-BASED IMMUNOTHERAPY AND PREDICTING THERAPEUTIC RESPONSE THERETO

(71) Applicants: Metaclipse Therapeutics Corporation, Atlanta, GA (US); Emory University, Atlanta, GA (US)

(72) Inventors: Periasamy Selvaraj, Lilburn, GA (US); Christopher Pack, Atlanta, GA (US); Sampath Ramachandiran, Duluth, GA (US); Shaker Reddy, Roswell, GA (US); Rammireddy Boomireddy, Johns Creek, GA (US)

(21) Appl. No.: 15/807,050

(22) Filed: Nov. 8, 2017

Related U.S. Application Data
(60) Provisional application No. 62/419,225, filed on Nov. 8, 2016.
(60) Provisional application No. 62/419,344, filed on Nov. 8, 2016.

(51) Int. Cl. G01N 33/574; A61K 38/17; A61K 38/20; A61K 39/395; A61K 47/69

(52) U.S. Cl. CPC G01N 33/57488; A61K 38/1774; A61K 38/208; A61K 39/39558; G01N 33/57415; G01N 2333/54; G01N 2333/57; G01N 2333/525; G01N 2333/55; G01N 2333/5434; A61K 47/6911

(57) ABSTRACT

Disclosed herein is a method for treating a subject having, or at risk of having, a triple negative breast cancer, comprising administering to the subject a therapeutically effective amount of an immunotherapeutic agent and a tumor membrane vesicle (TMV), wherein the TMV comprises a lipid membrane, and a B7-1 and/or IL-12 molecule anchored to the lipid membrane. Also disclosed is method for predicting the likelihood a human subject having a cancer will respond therapeutically to a TMV immunotherapy, wherein the method comprises obtaining a blood or serum sample from the subject; measuring the amount of a set of biomarkers in the sample, wherein the biomarkers include at least IFN-gamma, TNF-alpha, and IL-2, and wherein an increase in the level of the biomarkers as compared to a control indicates an increased likelihood the subject will respond therapeutically to the TMV immunotherapy.

### BACKGROUND

Immune dysfunction is associated with tumor progression and metastasis in cancer patients. Tumors evade the host immune system by numerous mechanisms such as suppression, anergy or deletion of effector T cells.

Recent developments in cancer therapies include the use of tumor membrane vesicles (TMVs) prepared from a patient's own tumor (see US2015/0071987). The tumor membrane vesicles are then modified by incorporating immunostimulatory agents (ISMs). Use of autologous tumor tissue from the patient incorporates the patient's unique immune signature into the vaccine design and overcomes the issue of heterogeneity within a single tumor and patient-to-patient variation in gene mutations.

Additional therapeutics for attacking tumors include the development of cancer immunotherapies. Immunotherapeutic agents do not directly attack the tumor, but boost the body's immune system to kill the cancer cells. The immune checkpoint blockade has elicited durable antitumor responses and long-term remissions in a subset of patients. Despite remarkable progress, current methods of checkpoint blockade therapy may limit the therapeutic benefits in many patients. In addition, the expense of the immunotherapy and uncertainty of immune response in many patients is still a major factor limiting immunotherapies. Prognostic biomarkers are needed for identifying patients likely to respond well to these cancer treatments and for the identification of methods of tracking and measuring a patient's immune response.

One particular type of breast cancer, triple negative breast cancer (TNBC), afflicts up to 50,000 women per year in the US, typically at a younger age than other breast cancers and with a poorer overall prognosis. This poor clinical outcome is attributed to a lack of a defined target, high patient-to-patient heterogeneity, and an aggressive phenotype. Even with conventional radiation and chemotherapy regimens, patients have poor prognosis, experiencing early, frequent relapses in comparison to other breast cancers. In addition, a high level of intratumoral as well as patient-to-patient heterogeneity is observed among triple negative patients, making it even more difficult to treat. See Gerlinger et. al., The New England Journal of Medicine, 366:883-92 (2012). Therapies effective for other cancers, even other breast cancers, frequently prove ineffective at treating TNBC. Thus, it is difficult to know whether a known anti-cancer therapy will be therapeutic in TNBC patients. TNBC is a clear area of significant unmet medical need, and new therapies that address patient-to-patient variation in tumor targets are critically required.

### SUMMARY

The compositions and methods disclosed herein address certain unmet needs in the cancer field. The TMV immunotherapy disclosed herein provides a personalized approach to treating TNBC, which suffers from a dearth of effective personalized therapies. Despite failure of numerous known anti-cancer agents to provide positive therapeutic outcomes for TNBC patients, the methods to treat TNBC using TMV immunotherapy disclosed herein resulted in surprisingly effective treatments with significant therapeutic outcomes. A method to predict whether a subject will respond therapeutically to a TMV immunotherapy is also provided herein.

Disclosed herein is a method for treating a subject having, or at risk of having, a triple negative breast cancer, comprising administering to the subject a therapeutically effective amount of an immunotherapeutic agent and a tumor membrane vesicle (TMV), wherein the TMV comprises a lipid membrane, and a B7-1 and/or IL-12 molecule anchored to the lipid membrane. In some embodiments, the TMV further comprises an antigen molecule anchored to the lipid membrane. In some embodiments, the immunotherapeutic agent comprises one or more of an anti-CTLA4 antibody, an anti-PD1 antibody, and an anti-PD-L1 antibody. In some embodiments, the treatment reduces metastasis or tumor size.

Also disclosed herein is a method for predicting the likelihood a subject having a cancer will respond therapeutically to a therapy administered to the subject, the therapy comprising administering a therapeutically effective amount of an immunotherapeutic agent and a tumor membrane vesicle (TMV), wherein the TMV comprises a lipid membrane, and a B7-1 and/or IL-12 molecule anchored to the lipid membrane, wherein the method for predicting comprises: obtaining a blood or serum sample from the subject; measuring protein expression levels of biomarkers in the sample, wherein the biomarkers include at least IFN-gamma, TNF-alpha, and IL-2, and wherein an increase in the levels of the biomarkers as compared to a control indicates an increased likelihood the subject will respond therapeutically to the therapy; and advising the subject of the increased likelihood the subject will respond therapeutically to the therapy when the relative levels of the biomarkers increase or advising the subject of the decreased likelihood the subject will respond therapeutically to the therapy when the relative levels of the biomarkers do not increase. In some embodiments, the TMV further comprises an antigen molecule anchored to the lipid membrane. In some embodiments, the antigen molecule is selected from HER-2, PSA, or PAP. In some embodiments, the TMV further comprises an adjuvant which, in some embodiments, is GM-CSF anchored to the lipid membrane. In some embodiments, the cancer is breast cancer, or in particular embodiments, triple-negative breast cancer. In some embodiments, the biomarkers further include IL-12, IL-18, IL-22, IL-23, or any combination thereof. In some embodiments, the immunotherapeutic agent comprises one or more of an anti-CTLA4 antibody, an anti-PD1 antibody, and an anti-PD-L1 antibody.

### BRIEF DESCRIPTION OF THE DRAWINGS

FIG. 1(A-C) is a set of graphs showing combination of 4T1 TMV+GPI-mB7-1/mIL-12 and anti-CTLA-4 mAb enhances long-term survival in a 4T1 tumor resection model. On Day 0, mice were challenged with 4T1 cells. On day 10, tumors were resected. On days 12 and 19, selected mice were vaccinated with TMVs locally via s.c injection. On days 12, 15, 19 and 22, selected mice were systemically i.p. injected with mAb therapy. On day 12, selected mice were treated with cyclophosphamide. FIG. 1A: Kaplan-Meier survival curves of control and anti-CTLA-4 mAb treated groups. Comparison of TMV+GPI-mB7-1/IL-12+anti-CTLA-4 mAb and anti-CTLA-4 mAb alone yields p=0.0074, n=5-10. Comparison of TMV+GPI-mB7-1/IL-12+anti-CTLA-4 mAb and TMV+GPI-mB7-1/IL-12+Mouse IgG yields p=0.0105, n=5-10. Log-rank (Mankel-Cox) test used for comparison analysis. FIG. 1B: Kaplan-Meier survival curves of control and a-PD-L1 mAb treated groups. FIG. 1C: Kaplan-Meier survival curves of control and cyclophosphamide (Cyp) treated groups. **p≤0.01. Data are representative of 2 independent experiments. The experiments in (A)-(C) are carried out simultaneously and for the purpose of clarity are divided into three graphs. Therefore, the controls in all three graphs are the same data.

FIG. 2 is a graph showing reduction in metastasis to the lung in mice receiving both 4T1 TMV containing GPI-mB7-1 and GPI-mIL-12 and anti-CTLA-4 mAb. For convenience, the immunostimulatory agents mB7-1 and mIL-12 are collectively referred to as “ISM.” The immunotherapy and tumor challenge protocol was as follows: Three weeks prior to challenge, mice were vaccinated with 100 μg TMVs incorporated with GPI-ISMs (modified TMVs). One week prior to challenge, another 100 μg modified TMV boost was administered. Mice were then challenged with 20,000 4T1 cells and administered mAb therapy. Anti-CTLA-4 mAb (Clone 9D9) was given systemically via i.p. injection 1 day following 4T1 tumor challenge and subsequently 3 days later for a total of 4 doses: dose 1 (200 μg), 2 (100 μg), 3 (100 μg), and 4 (100 μg) in 200 μl PBS). 27-28 days later, mice were sacrificed and lung metastasis was evaluated. Metastasis was evaluated using a clonogenic assay at day 27-28 post orthotopic challenge. **p<0.01, n=3-5. Data are representative of 2 independent experiments. Group 1: PBS; 2: 4T1 TMV only; 3: 4T1 TMV+GPI-ISM; 4: 4T1 TMV+GPI-ISM+aCTLA-4 mAb; 5: 4T1 TMV+GPI-ISM+mouse IgG; 6: aCTLA-4 mAb alone; 7: 4T1 TMV+GPI-ISM+aPD-L1 mAb; 8: 4T1 TMV+GPI-ISM+rat IgG; 9: aPD-L1 mAb alone.

FIG. 3 is a graph showing reduction in metastasis to the lung in mice receiving both 4T1 TMV containing GPI-mB7-1 and GPI-mIL-12 and anti-CTLA-4 mAb post tumor challenge. For convenience, the immunostimulatory agents GPI-mB7-1 and GPI-mIL-12 are collectively referred to as “ISM.” The tumor challenge, resection, and immunotherapy protocol was as follows: On Day 0, mice were challenged with 4T1 cells. On Day 10, tumors were resected. On Days 12 and 19, mice were TMV-vaccinated. Anti-CTLA-4 mAb (Clone 9D9) was given i.p. 1 day following TMV immunotherapy and subsequently 3 days later for a total of 4 doses: dose 1 (200 μg), 2 (100 μg), 3 (100 μg), and 4 (100 μg) in 200 μl PBS). Metastasis was evaluated using a clonogenic assay at day 35-36 post orthotopic challenge. *p<0.05, n=4-5. Data are representative of 2 independent experiments. Group 1: PBS; 2: 4T1 TMV+GPI-ISM; 3: 4T1 TMV+GPI-ISM+aCTLA-4 mAb; 4: aCTLA-4 mAb alone; 5: tumor challenge with no resection.

FIG. 4 is a graph showing depletion of CD8 T cells, but not CD4 T cells, abrogates impact of immunotherapy upon 4T1 metastasis. The immunotherapy, cell depletion, and tumor challenge protocol were as follows: three weeks prior to challenge, mice were vaccinated with 100 μg modified TMVs. One week prior to challenge, another 100 modified μg TMV boost was administered. Anti-mouse CD4 (Clone GK1.5) and CD8 (CloneYTS 169.4) antibodies were used for cell depletion (500 μg dose given i.p. once in 200 μl PBS one day prior to tumor challenge). Mice were then challenged with 4T1 cells on Day 0. On Day 20, mice were sacrificed to evaluate lung metastasis. Anti-CTLA-4 mAb (Clone 9D9) was given i.p. 1 day following TMV immunotherapy and subsequently every 3 days for a total of 4 doses as above. Metastasis was evaluated using a clonogenic assay at day 20 post orthotopic challenge with 10⁴ 4T1 cells. **p<0.01, n=4-5. Data are representative of 2 independent experiments. Group 1: PBS; 2: 4T1 TMV+GPI-ISMs; 3: 4T1 TMV+GPI-ISMs+aCTLA-4 mAb; 4: 4T1 TMV+GPI-ISMs+aCTLA-4 mAb+aCD4 mAb; 5: 4T1 TMV+GPI-ISMs+aCTLA-4 mAb+aCD8 mAb. For convenience, the immunostimulatory agents B7-1 and IL-12 are collectively referred to as “ISM.”

FIG. 5(A-B) is a set of graphs showing administration of anti-CTLA-4 mAb augments immunotherapy-induced tumor-specific immune responses. Briefly, mice were immunized twice 14 days apart with 4T1 TMV, 4T1 TMV+GPI-ISM+GPI-hHER-2ED, or 4T1 TMV+GPI-ISM+GPI-hHER-2ED+anti CTLA-4 mAb (wherein hHER-2ED refers to human HER-2 Extracellular Domain). Spleens were removed on day 10 after the final immunization and processed into a single cell suspension. FIG. 5A: Enhanced HER-2 peptide-specific IFN-gamma ELISPOT response in mice receiving combination therapy. The HER-2 p63-71 peptide specific immune response was evaluated in an ELISPOT assay for IFN-γ secreting cells. Spleen cells at 0.5×10⁶ cells/well were stimulated for 48 hours with 1 μM HER-2 p63-71 peptide. Group 1: 4T1 TMV only; 2: 4T1 TMV+GPI-ISM+GPI-hHER-2; 3: 4T1 TMV+GPI-ISM+GPI-hHER-2+aCTLA-4 mAb. FIG. 5B: Increased 4T1 cell-specific IFN-gamma ELISPOT response in combination therapy mice. Mice were immunized as above, but GPI-hHER-2ED was not incorporated into the TMV. Spleen cells at 0.5×10⁶ cells/well were stimulated for 48 hours with 5×10⁴ mitomycin C treated 4T1 cells. Group 1: PBS; 2: 4T1 TMV only; 3: 4T1 TMV+GPI-ISM; 4: 4T1 TMV+GPI-ISM+aCTLA-4 mAb. *p<0.05, **p<0.01. n=3. Data are representative of 2 independent experiments. For convenience, the immunostimulatory agents B7-1 and IL-12 are collectively referred to as “ISM.”

FIG. 6(A-G) is a set of graphs showing co-administration of anti-CTLA-4 mAb with immunotherapy increases several key immune mediators in serum. Mice (n=5) were immunized three times 14 days apart with PBS, anti-CTLA-4 mAb, 4T1 TMV vaccine, or 4T1 TMV vaccine+anti-CTLA-4 mAb. Serum was collected and pooled 5 days after the final immunization, and an eBioscience 36-Plex Luminex assay was performed by Charles River Laboratories. Serum cytokines having particularly increased levels in response to treatments included IFN-γ (FIG. 6A), TNFα (FIG. 6B), IL-2 (FIG. 6C), IL-12 (FIG. 6D), IL-18 (FIG. 6E), IL-22 (FIG. 6F), and IL-23 (FIG. 6G). Group 1: PBS; 2: anti-CTLA-4 mAb; 3: 4T1 TMV vaccine; 4: 4T1 TMV vaccine+anti-CTLA-4 mAb.

FIG. 7(A-E) is a set of graphs showing biological activity of ISMs incorporated onto TMV prepared from human TNBC cell lines or breast cancer tumor tissue. FIG. 7A: Human PBMCs were activated with OKT-3 anti-TCR (anti-CD3) mAb 24 h prior to stimulation. TMVs with/without GPI-IL-12 (40 ug/ml) from various TNBC cell lines were then used to stimulate activated PBMC for 48 h. IFN-gamma was then measured in the culture supernatant by ELISA. TMVs for groups 1, 3, 5, and 7: TMV without GPI-IL-12; groups 2, 4, 6, and 8: TMV+GPI-IL-12. TMV sources are: groups 1 and 2: MDA-MB-231; groups 3 and 4: MDA-MB-453; groups 5 and 6: BT-549; groups 7 and 8: HCC-1187. TMV were prepared from respective human TNBC cell lines described above. FIG. 7B: Human NK-92 cells were stimulated with TMVs containing incorporated GPI-IL-12 (40 ug/ml TMV) from various human TNBC cell lines above for 48 h. TMVs and TMV sources are as in FIG. 7A. IFN-gamma was then measured in the culture supernatant by ELISA. FIG. 7C: TMVs were prepared from de-identified human breast cancer tumor samples acquired from a tumor tissue bank. TMVs were either unmodified: groups 1, 3, and 5 or modified with GPI-B7-1 and GPI-IL-12 in groups 2, 4, and 6. FIG. 7D: Human NK-92 cells were stimulated with patient-derived TMVs containing incorporated GPI-IL-12 (40 ug/ml TMV) patient-derived breast cancer tumor tissue for 48 h. FIG. 7E: Human Jurkat E6.1 cells were stimulated with patient-derived TMVs with/without GPI-B7-1 (40 ug/ml TMV) from patient-derived breast cancer tumor tissue in combination with anti-CD3 for 24 h. Human TMVs were pre-coated on the tissue culture plates overnight @4° C. prior to the assay. IFN-gamma and IL-2 in the culture supernatants were measured by sandwich ELISA. IL-2 levels were undetectable in Jurkat E6.1 or PBMC stimulated with anti-CD3 alone. PMA+Ionomycin was used as positive control for these assays. TMVs for groups 1, 3, and 5: TMV without GPI-B7-1; groups 2, 4, and 6: TMV+GPI-B7-1. TMV sources are: groups 1 and 2: tumor sample 1; groups 3 and 4: tumor sample 2; groups 5 and 6: tumor sample 3.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular nanoparticle is disclosed and discussed and a number of modifications that can be made to the nanoparticle are discussed, specifically contemplated is each and every combination and permutation of the nanoparticle and the modifications that are possible unless specifically indicated to the contrary. Thus, if a class of nanoparticles A, B, and C are disclosed as well as a class of nanoparticles D, E, and F and an example of a combination nanoparticle, or, for example, a combination nanoparticle comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods. It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” or “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymphatic systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

As used herein, the term “anchored to the lipid membrane” refers to the insertion of an exogenous polypeptide such as B7-1, B7-2 and/or IL-12 at the exterior of the lipid membrane surface. The term “anchored to the lipid membrane” does not refer to endogenous polypeptides naturally expressed at a cell's surface.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics.

“Immunotherapeutic agent” refers to any composition that has a beneficial biological effect by way of increasing, promoting, inducing, or stabilizing an immune response. In some embodiments, an immunotherapeutic agent facilitates an anti-tumor and/or an anti-metastasis immune response. Beneficial biological effects include both therapeutic effects, e.g., treatment of a disorder or other undesirable physiological condition, and prophylactic effects, e.g., prevention of a disorder or other undesirable physiological condition (e.g., cancer).

“Therapeutically effective amount” or “therapeutically effective dose” of a composition (e.g. a composition comprising an agent) refers to an amount that is effective to achieve a desired therapeutic result. In some embodiments, a desired therapeutic result is the reduction in tumor size or metastasis. Therapeutically effective amounts of a given agent will typically vary with respect to factors such as the type and severity of the disorder or disease being treated and the age, gender, weight, and general condition of the subject. Thus, it is not always possible to specify a quantified “therapeutically effective amount.” However, an appropriate “therapeutically effective amount” in any subject case may be determined by one of ordinary skill in the art using routine experimentation. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. It is understood that, unless specifically stated otherwise, a “therapeutically effective amount” of a therapeutic agent can also refer to an amount that is a prophylactically effective amount. In some instances, a desired biological or medical response is achieved following administration of multiple dosages of the composition to the subject over a period of days, weeks, or years.

“Treat,” “treating,” “treatment,” and grammatical variations thereof as used herein, include the administration of a composition with the intent or purpose of partially or completely, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease.

“Specifically binds” when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

### Tumor Membrane Vesicles (TMVs)

The tumor membrane vesicles (TMVs) used in the methods herein are described in PCT patent application PCT/US2013/024355 (WO2013/116656), the contents of which are herein incorporated by reference in its entirety.

The TMV is a particle formed from cell membrane material obtained from a tumor (e.g., surgically resected patient tumor tissue). Because the TMV contains tumor cell membrane material, the TMV contains tumor associated molecules and/or tumor-specific molecules (e.g., antigens). These tumor-specific antigens can activate the subject's immune system by active immunization with tumor antigens. Thus, TMVs represent a personalized, tissue-derived strategy for treating tumors in a subject.

The TMV contains a lipid membrane comprised of tumor associated molecules and/or tumor specific molecules (e.g., antigens). Further, additional molecules not specifically derived from a tumor or tumor sample can be attached to the lipid membrane. These additional molecules include one or more immunostimulatory agents, one or more antigens, and one or more additional anti-tumor compounds (e.g., anti-neoplastic agent). The lipid membrane may be in the form of a monolayer or bilayer (e.g., a phospholipid monolayer or phospholipid bilayer), or mixtures thereof.

Typically, the TMV contains an immunostimulatory agent (ISM) attached to the lipid membrane of the TMV. As used herein, an “immunostimulatory agent” is any molecule that, when attached to a TMV, can stimulate or co-stimulate an anti-tumor immune response. TMVs containing membrane-attached immunostimulatory agents deliver molecules which stimulate immune responses, as well as patient-specific tumor antigens, and activate immune cells to promote an anti-tumor immune response.

In some embodiments, the immunostimulatory agent is B7-1 (also known as CD80), B7-2 (also known as CD86), IL-12, GM-CSF, IL-2 or combinations thereof. In some embodiments, the immunostimulatory agent is B7-1, B7-2, IL-12, or combinations thereof. In some embodiments, the immunostimulatory agent is B7-1, IL-12, or combinations thereof. In some embodiments, the TMV includes one immunostimulatory agent or, alternatively, two or more immunostimulatory agents.

In some embodiments, the immunostimulatory agent B7-1 comprises an amino acid sequence of SEQ ID NO: 1 or a fragment thereof. In some embodiments, the immunostimulatory agent B7-1 comprises an amino acid sequence of SEQ ID NO: 2 or a fragment thereof. In some embodiments, the immunostimulatory agent B7-1 comprises an amino acid sequence of SEQ ID NO: 3 or a fragment thereof. In some embodiments, the immunostimulatory agent B7-1 is that identified in one or more publicly available databases as follows: HGNC: 1700 Entrez Gene: 941 Ensembl: ENSG00000121594 OMIM: 112203 UniProtKB: P33681. In some embodiments, the immunostimulatory agent B7-1 comprises a polypeptide sequence having about 70% or greater, about 75% or greater, about 80% or greater, about 85% or greater, about 90% or greater, about 95% or greater, or about 98% or greater homology with SEQ ID NO: 1, SEQ ID NO:2, or SEQ ID NO:3.

In some embodiments, the immunostimulatory agent B7-2 comprises the amino acid sequence of SEQ ID NO:4, or a fragment thereof. In some embodiments, the immunostimulatory agent B7-2 is that identified in one or more publicly available databases as follows: HGNC: 1705 Entrez Gene: 942 Ensembl: ENSG00000114013 OMIM: 601020 UniProtKB: P42081. In some embodiments, the immunostimulatory agent B7-2 used comprises a polypeptide sequence having about 70% or greater, about 75% or greater, about 80% or greater, about 85% or greater, about 90% or greater, about 95% or greater, or about 98% or greater homology with SEQ ID NO: 4.

In some embodiments, IL-12 comprises IL-12a and IL-12b. In some embodiments, the immunostimulatory agent IL-12 comprises the sequence of SEQ ID NO: 5, or a fragment thereof. In some embodiments, the immunostimulatory agent IL-12 is that found in one or more publicly available databases as follows: HGNC: 5969 Entrez Gene: 3592 Ensembl: ENSG00000168811 OMIM: 161560 UniProtKB: P29459. In some embodiments, the immunostimulatory agent IL-12 comprises a polypeptide sequence having about 70% or greater, about 75% or greater, about 80% or greater, about 85% or greater, about 90% or greater, about 95% or greater, or about 98% or greater homology with SEQ ID NO: 5.

In some embodiments, the immunostimulatory agent IL-12 comprises the sequence of SEQ ID NO: 6, or a fragment thereof. In some embodiments, the immunostimulatory agent IL-12 is that found in one or more publicly available databases as follows: HGNC: 5970 Entrez Gene: 3593 Ensembl: ENSG00000113302 OMIM: 161561 UniProtKB: P29460. In some embodiments, the immunostimulatory agent IL-12 comprises a polypeptide sequence having about 70% or greater, about 75% or greater, about 80% or greater, about 85% or greater, about 90% or greater, about 95% or greater, or about 98% or greater homology with SEQ ID NO: 6. The immunostimulatory agent (e.g., B7-1, B7-2 and/or IL-12) is anchored to the lipid membrane of the vesicle. Other molecules, for instance, an antigen molecule such as a tumor specific antigen or cancer marker, can also be anchored to the lipid membrane of the vesicle. As used herein, the term “anchored to the lipid membrane” refers to the insertion of an exogenous polypeptide such as B7-1, B7-2 and/or IL-12 at the exterior of the lipid membrane surface. The term “anchored to the lipid membrane” does not refer to endogenous polypeptides naturally expressed at a cell's surface.

In some embodiments, the immunostimulary molecule (e.g., B7-1, B7-2 and/or IL-12), antigen molecule, or other molecules (e.g., tumor-specific proteins) can be anchored onto the membrane of the TMV through a variety of linkages, such as lipid palmatic acid, biotin-avidin interaction, or a glycosylphosphatidylinositol (GPI)-anchor. Accordingly, polypeptides described herein can be anchored to a lipid membrane, or TMV membrane via a glycosylphosphatidylinositol (GPI)-anchor. For example, glycosyl phosphatidylinositol anchored B7-1 (GPI-B7-1) molecules have been incorporated onto tumor cells and isolated tumor cell membranes to provide costimulation for allogenic T cell proliferation. See Nagarajan et. al., Vaccine, 24(13):2264-74 (2006), U.S. Published Patent Application No. US 2007/0243159, Bozeman et al., Front Biosci., 15:309-320 (2010). As used herein, a GPI-anchored molecule (for instance, B7-1) is preceded by “GPI-” (e.g., GPI-B7-1).

GPI-anchored polypeptides can be created through the addition of a GPI anchor signal sequence to the polypeptide. A GPI anchor signal sequence is a sequence that directs GPI anchor addition to the polypeptide. One example of a GPI anchor signal sequence that may be added to a polypeptide is SEQ ID NO: 11, a CD59 GPI anchor signal sequence. Accordingly, in some embodiments, the immunostimulatory agent, antigen, or other molecules attached to the lipid membrane include a GPI anchor signal sequence.

A number of proteins commonly expressed by cells are attached to the cell membrane via a GPI-anchor. These proteins are post-translationally modified at their carboxy terminus to express this glycosylated moiety which is synthesized in the endoplasmic reticulum. These naturally expressing GPI-anchored molecules are widely distributed in mammalian cells and serve a host of different cellular functions, such as cell adhesion, enzymatic activity, and complement cascade regulation. Naturally occurring GPI-anchored proteins lack a transmembrane and cytoplasmic domain that otherwise anchor membrane proteins. The GPI-anchor consists of a glycosylated moiety attached to phosphatidylinositol containing two fatty acids. The phosphatidylinositol portion, as well as an ethanolamine which is attached to the C-terminal of the extracellular domain of the membrane proteins, anchor the molecule to the cell membrane lipid bilayer.

In order to exploit this natural linkage using recombinant DNA techniques, the transmembrane and cytoplasmic domains of a transmembrane surface protein need only be replaced by the signal sequence for GPI-anchor attachment that is found at the hydrophobic C-terminus of GPI-anchored protein precursors. This method may be used to generate GPI-anchored proteins is not limited to membrane proteins; attaching a GPI-anchor signal sequence to a secretory protein also converts the secretory protein to a GPI-anchored form. The method of incorporating the GPI-anchored proteins onto isolated cell surfaces or TMVs is referred to here as protein transfer.

GPI-anchored molecules can be incorporated onto lipid membranes spontaneously. GPI-anchored proteins can be purified from one cell type and incorporated onto cell membranes of a different cell type. GPI-anchored proteins can be used to customize the lipid membranes disclosed herein. Multiple GPI-anchored molecules can be simultaneously incorporated onto the same cell membrane. The amount of protein attached to the TMV can be controlled by simply varying the concentration of the GPI-anchored molecules to be incorporated onto membranes. A significant advantage of this technology is the reduction of time in preparing cancer vaccines from months to hours. These features make the protein transfer approach a more viable choice for the development of cancer vaccines for clinical settings. The molecules incorporated by means of protein transfer retain their functions associated with the extracellular domain of the native protein. Cells and isolated membranes can be modified to express immunostimulatory agents. In certain embodiments, the disclosure contemplates that the GPI-anchored molecules are incorporated onto the surface of TMVs by this protein transfer method. GPI-anchored proteins attached to the surface of TMVs are used for an array of functions, at least including immunostimulation, co-stimulation, boosting immune responses, generating long term memory, etc., thereby enhancing the capacity to function as a targeted therapy for cancer treatment.

The GPI-B7-1 incorporation (by protein transfer) was stable up to 7 days on isolated membranes at 37° C., and frozen membranes can be used up to 3 years of storage at −80° C., rendering stability and storage a nonissue. These studies show that membrane-based TMV vaccines are more suitable to stably express GPI-anchored molecules than intact cells, which significantly lose expression of the GPI-anchored molecules within about 24 hours.

The protein transfer strategy provides advantages over other immunotherapies for cancer vaccine development. Protein transfer allows a protein to be added either singularly or in a combinatory manner to the TMV surface. This approach does not require the establishment of tumor cells, unlike for gene transfer. This GPI-mediated approach by protein transfer may be used for an array of molecules, such as immunostimulatory agents (e.g., B7-1, B7-2, GM-CSF, IL-2, and IL-12). Further, immunostimulatory agents attached to the TMV via a GPI-anchor can exert their effector functions locally at the vaccination site with reduced or no risk of systemic toxicity.

In some embodiments, the TMV further comprises an antigen molecule. The antigen molecule can be attached to the lipid membrane of the TMV, for example by a GPI anchor. Thus, in some embodiments, the antigen molecule is modified to include a GPI-anchor amino acid sequence.

In some embodiments, the TMV further comprises two or more antigen molecules. For example, the TMV can comprise at least two, at least three, at least four, at least five, at least six, at least seven, at least eight, at least nine, at least ten, or more antigen molecules.

In some embodiments, the antigen molecule in the tumor membrane vesicle (TMV) can be HER-2, MKI67, prostatic acid phosphatase (PAP), prostate-specific antigen (PSA), prostate-specific membrane antigen, early prostate cancer antigen, early prostate cancer antigen-2 (EPCA-2), BCL-2, MAGE antigens such as CT7, MAGE-A3 and MAGE-A4, ER 5, G-protein coupled estrogen receptor 1, CA15-3, CA19-9, CA 72-4, CA-125, carcinoembryonic antigen, CD20, CD31, CD34, PTPRC (CD45), CD99, CD 117, melanoma-associated antigen (TA-90), peripheral myelin protein 22 (PMP22), epithelial membrane proteins (EMP-1, -2, and -3), HMB-45 antigen, MART-1 (Melan-A), S100A1, S100B and gp 100:209-217(210M), MUC-1, mucin antigens TF, Tn, STn, glycolipid globo H antigen, or any combination thereof. Typically, the antigen is the human form. HER-2, or Human Epidermal Growth Factor Receptor 2, refers to the human protein encoded by the ERBB2 gene that has been referred to as Neu, ErbB-2, CD340 (cluster of differentiation 340) or p185. See Coussens et al, Science, 230 (4730): 1132-9 (1985).

In some embodiments, the antigen molecule comprises HER-2 or a fragment thereof. In some embodiments, the HER-2 comprises an amino acid of SEQ ID NO: 7 or a fragment thereof. In some embodiments, the antigen molecule HER-2 comprises an amino acid sequence identical to SEQ ID NO: 8 or a fragment thereof. In some embodiments, the antigen molecule HER-2 comprises an amino acid sequence of, SEQ ID NO: 9 or a fragment thereof. In some embodiments, the antigen molecule HER-2 is that identified in one or more publicly available databases as follows: HGNC: 3430 Entrez Gene: 2064 Ensembl: ENSG00000141736 OMIM: 164870 UniProtKB: P04626. In some embodiments, the antigen molecule HER-2 comprises a polypeptide sequence having about 70% or greater, about 75% or greater, about 80% or greater, about 85% or greater, about 90% or greater, about 95% or greater, or about 98% or greater homology with SEQ ID NO: 7, SEQ ID NO: 8, or SEQ ID NO: 9.

### Immunotherapeutic Agents

The immunotherapeutic agent can be any agent that, when administered with a TMV comprising a lipid membrane and a B7-1 and/or IL-12 molecule anchored to the lipid membrane, enhances an anti-tumor and/or an anti-metastasis immune response.

In some embodiments, the immunotherapeutic agent comprises an immune checkpoint inhibitor (ICI). Immune checkpoint inhibitors (sometimes referred to as checkpoint blockade inhibitors (CBI) or checkpoint inhibitors) can increase the effectiveness of overall T cell anti-tumor immunity. ICIs block certain activities of particular proteins produced by immune cells (e.g., T cells) and cancer cells that keep immune cells “in check,” or in other words, prevent immune cells from attacking or killing a cell (e.g., cancer cell). When ICIs block checkpoint proteins, immune cells such as T cells can more effectively mount a response to the cancer cell.

In some embodiments, the immunotherapeutic agent comprises an antibody, particularly an antibody having ICI function. In some embodiments, the immunotherapeutic agent comprises an anti-CTLA4 antibody, an anti-PD1 antibody, an anti-PDL1 antibody, or any combination thereof.

In some embodiments, the immunotherapeutic agent comprises an anti-CTLA4 antibody. In some embodiments, the anti-CTLA4 antibody comprises abatacept, belatacept, ipilimumab, tremelimumab, or any combination thereof. In some embodiments, the anti-CTLA4 antibody is ipilimumab. An anti-CTLA4 antibody is defined herein as a polypeptide capable of specifically binding a CTLA4 polypeptide.

In some embodiments, the immunotherapeutic agent comprises an anti-PDL1 antibody. In some embodiments, the anti-PDL1 antibody comprises atezolizumab, durvalumab, avelumab, or any combination thereof. In some embodiments, the anti-PDL1 antibody is atezolizumab (MPDL3280A) (Roche), durvalumab (MEDI4736), avelumab (MS0010718C), or any combination thereof. An anti-PDL1 antibody is defined herein as a polypeptide capable of specifically binding a PDL1 polypeptide.

In some embodiments, the immunotherapeutic agent comprises a programmed death protein 1 (PD-1) inhibitor, programmed death protein ligand 1 or 2 inhibitor, or any combination thereof. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech).

In some embodiments, the immunotherapeutic agent comprises an anti-PD1 antibody. In some embodiments, the anti-PD1 antibody is nivolumab, pembrolizumab, or any combination thereof. An anti-PD-1 antibody is defined herein as a polypeptide capable of specifically binding PD-1 polypeptide.

### Anti-Neoplastic Agents

Compositions herein can include one or more anti-neoplastic agents. In some embodiments, the anti-neoplastic agent can include Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Interferon Alfa-2b, Recombinant, Intron A (Recombinant Interferon Alfa-2b), Iodine I 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), and combinations thereof.

### Methods for Treating

Disclosed herein is a method for treating a subject having, or at risk of having, breast cancer, comprising administering to the subject a therapeutically effective amount of an immunotherapeutic agent and a tumor membrane vesicle (TMV), wherein the TMV comprises a lipid membrane and an immunostimulatory agent anchored to the lipid membrane. The TMV used in the methods can be any herein disclosed TMV.

Also disclosed herein is a method for treating a subject having, or at risk of having, a triple negative breast cancer, comprising administering to the subject a therapeutically effective amount of an immunotherapeutic agent and a tumor membrane vesicle (TMV), wherein the TMV comprises a lipid membrane, and a B7-1 and/or IL-12 molecule anchored to the lipid membrane.

The subject can be any mammalian subject, for example a human, dog, cow, horse, mouse, rabbit, etc. In some embodiments, the subject is a primate, particularly a human. The subject can be a male or female of any age, race, creed, ethnicity, socio-economic status, or other general classifiers.

The subject has, or is at risk of having, breast cancer. A subject can be at risk of having breast cancer by being genetically predisposed to having breast cancer. For example, and without limitation, breast cancer genetic predisposition can arise from mutations in one or both alleles of BRCA1, BRCA2, CHEK2, ATM, BRIPI, PALB2, RAD50, RAD51B, RAD51C, RAD51D, XRCC2, CDH1, TP53, PTEN, STK11/LKB1, FGFR2, p53, NBS1, BARDI, MRE11, FANCA, FANCC, and FANCM, among other genetic biomarkers of breast cancer. A subject at risk of having breast cancer includes a subject previously diagnosed with breast cancer and subsequently clinically determined to be in partial or complete remission, and includes a subject previously diagnosed with breast cancer that has undergone a procedure (e.g. surgery) to remove some or all of a breast cancer tumor.

In some embodiments, the cancer is a triple negative breast cancer. Triple negative breast cancer (TNBC) is defined as a cancer or tumor lacking expression of estrogen receptor, progesterone receptor, and HER-2 protein. TNBC represents one of the most challenging cancers for developing an effective therapy post tumor resection due to lack of a therapeutic target. Even with conventional radiation and chemotherapy regimens, patients can have poor prognosis, experiencing early, frequent relapses in comparison to other breast cancers. In addition, a high level of intratumoral as well as patient-to-patient heterogeneity is observed among triple negative patients, making it even more difficult to treat. See Gerlinger et. al., N. Engl. J. Med., 366:883-92 (2012). Therapies effective for other cancers, even other breast cancers, frequently prove ineffective at treating TNBC. Thus, it is difficult to predict therapeutic outcomes in TNBC for known anti-cancer agents and treatment regimens. TNBC is a clear area of significant unmet medical need, and new therapies that address patient-to-patient variation in tumor targets are critically required. See Peddi et. al., Int. J. Breast Cancer, 217185 (2012). In some embodiments, the triple negative breast cancer is a metastatic triple negative breast cancer.

The TMV used in the methods comprises a lipid membrane and an immunostimulatory agent anchored to the lipid membrane. The immunostimulatory agent can be any herein disclosed immunostimulatory agent. In some embodiments, the immunostimulatory agent can comprise a full-length polypeptide or, alternatively, can comprise an immunostimulatory portion of full-length immunostimulatory agent.

In some embodiments, the immunostimulatory agent comprises a B7-1, B7-2, or IL-12 molecule. In some embodiments, the immunostimulatory agent comprises a B7-1 or IL-12 molecule. In some embodiments, only a B7-1 molecule is selected. In some embodiments, only a IL-12 molecule is selected. In some embodiments, the TMV comprises both a B7-1 and a IL-12 molecule anchored to the lipid membrane. In some embodiments, the TMV further comprises one or more additional immunostimulatory agents, for instance, B7-2, GM-CSF, and/or IL-2.

In some embodiments, the immunostimulatory agent can be anchored onto the membrane of the TMV through a variety of linkages, such as via lipid palmatic acid, biotin-avidin interaction, or a glycosylphosphatidylinositol (GPI)-anchor. In some embodiments, the GPI-anchored immunostimulatory agent (e.g., IL-12) has reduced liver toxicity as compared to the soluble form of the molecule.

In some embodiments, the TMV further comprises an antigen molecule anchored to the lipid membrane. The antigen molecule can comprise any herein disclosed antigen molecule. The entire antigen molecule or, alternatively, an antigenic portion of the antigen molecule can be used. In some embodiments, the antigen is a protein, or alternatively, an antigenic fragment of a protein. In some embodiments, the TMV contains an antigen molecule comprising HER-2, PSA, or PAP. Optionally, the antigen molecule is HER-2. In some embodiments, the antigen molecule is the extracellular domain of HER-2 which includes the peptide consisting essentially of amino acids 63-71 of human HER-2 (the “p63-71” peptide) having a sequence of SEQ ID NO: 10.

In some embodiments, the antigen molecule may be anchored onto the membrane of the TMV through a variety of linkages, such as via lipid palmatic acid, biotin-avidin interaction, or a glycosylphosphatidylinositol (GPI)-anchor.

The methods comprise administering to the subject a therapeutically effective amount of an immunotherapeutic agent. As such, a combination therapy comprising a TMV and an immunotherapeutic agent is administered. Administering a combination of an immunotherapeutic agent with TMV immunotherapy can significantly enhance immune responses and increases response rates. This can be shown at least by induction of anti-tumor immunity and the infiltration of immune cells into TNBC tumor tissue, which is a positive prognostic indicator. See van Rooijen et. al., Pharmacol. Ther., 156:90-101 (2015). For instance, TMV and immunotherapeutic agent combination therapy generates protective immunity, reduces metastasis, and prolongs survival in the aggressive 4T1 model of TNBC. 4T1 is a mammary carcinoma tumor model derived from a spontaneous tumor in BALB/c mice and shares many characteristics with naturally occurring human breast cancer. Additionally, the inclusion of an antigen molecule in the TMV and immunotherapeutic agent combination therapy can aid in disrupting metastasis.

The immunotherapeutic agent can be any herein disclosed immunotherapeutic agent. In some embodiments, the immunotherapeutic agent comprises an immune checkpoint inhibitor (ICI). In some embodiments, the immunotherapeutic agent comprises an antibody, particularly an antibody having ICI function. In some embodiments, the immunotherapeutic agent can include one or more of an anti-CTLA4 antibody, an anti-PD1 antibody, an anti-PDL1 antibody, or any combination thereof.

In some embodiments, the anti-CTLA4 antibody can include abatacept, belatacept, ipilimumab, tremelimumab, or any combination thereof. In some embodiments, the anti-CTLA4 antibody is ipilimumab. In some embodiments, the anti-PDL1 antibody can include atezolizumab, durvalumab, avelumab, or any combination thereof. In some embodiments, the anti-PDL1 antibody is atezolizumab (MPDL3280A) (Roche), durvalumab (MEDI4736), avelumab (MS0010718C), or any combination thereof. In some embodiments, the PD-1 inhibitor can include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech). In some embodiments, the anti-PD1 antibody is nivolumab, pembrolizumab, or any combination thereof. In some embodiments, the administering step can include substitution of an anti-neoplastic agent for the immunotherapeutic agent. In some embodiments, the administering step can include administering the immunotherapeutic agent in combination with an anti-neoplastic agent. The anti-neoplastic agent can be any herein disclosed anti-neoplastic agent.

In some embodiments, the method further comprises administering an adjuvant. The adjuvant can be administered prior to, concurrent with, or subsequent to administration of the TMV and the immunotherapeutic agent. In some embodiments, the adjuvant is GM-CSF, or any biocompatible FDA-approved adjuvant. In some embodiments, the adjuvant comprises IL-2, ICAM-1, GM-CSF, flagellin, unmethylated, CpG oligonucleotide, lipopolysaccharides, or lipid A. The adjuvant can be in a form separate from the TMV or can be anchored to the lipid membrane of the TMV (by, for example, via a GPI anchor). In some embodiments, the TMV further comprises an adjuvant anchored to the lipid membrane wherein the adjuvant and antigen molecule are not the same molecule.

The administering step can include any method of introducing the immunotherapeutic agent and TMV into the subject appropriate for the combination therapy formulation. The administering step can include at least one, two, three, four, five, six, seven, eight, nine, or at least ten dosages. The administering step can be performed before the subject exhibits disease symptoms (e.g., prophylactically), or during or after disease symptoms occur or after other treatment modalities such as surgery, chemotherapy, and radiation. The administering step can be performed prior to, concurrent with, or subsequent to administration of other agents to the subject. The administering step can be performed with or without co-administration of additional agents (e.g., additional immunostimulatory agents, anti-neoplastic agents).

The method can include systemic administration of the immunotherapeutic agent and TMV (e.g., injection into the circulatory or lymphatic systems). Alternatively, the method can include local administration of the immunotherapeutic agent and TMV. For example, the immunotherapeutic agent and TMV can be administered locally to a tumor or an area near a tumor. In some embodiments, the immunotherapeutic agent and TMV are administered to areas of the subject comprising tumors. Alternatively, the method can include systemic administration of the immunotherapeutic agent and local administration of the TMV.

In some embodiments, the treatment comprising administering to a subject a therapeutically effective amount of an immunotherapeutic agent and a TMV reduces metastasis of triple negative breast cancer. In some embodiments, the treatment reduces the size of a tumor. In some embodiments, the treatment does not result in substantial liver toxicity.

In some embodiments, the method can further include administering to the subject a therapeutically effective amount of an immunotherapeutic agent and TMV and a pharmaceutically acceptable excipient. Suitable excipients include, but are not limited to, salts, diluents, binders, fillers, solubilizers, disintegrants, preservatives, sorbents, and other components. Also disclosed herein is a medicament comprising a pharmaceutically effective amount of immunotherapeutic agent and a TMV, wherein the TMV comprises a lipid membrane and an immunostimulatory agent anchored to the lipid membrane.

In some embodiments, the method includes administering to the subject a medicament comprising a pharmaceutically effective amount of immunotherapeutic agent and a TMV, wherein the TMV comprises a lipid membrane and an immunostimulatory agent anchored to the lipid membrane. Generally, the medicament comprises a pharmaceutically acceptable excipient and a pharmaceutically effective amount of an immunotherapeutic agent and a TMV.

### Methods for Predicting

Also disclosed herein is a method for predicting the likelihood a mammal will respond therapeutically to a TMV therapy, the method comprising measuring in a blood or serum sample of the mammal an amount of a set of biomarkers comprising IFN-gamma, TNF-alpha, and 1-2, and predicting the likelihood a mammal will respond therapeutically to a therapy based on an increased amount of the biomarkers compared to a control.

Also disclosed is a method for predicting the likelihood a subject having a cancer will respond therapeutically to a therapy administered to the subject, the therapy comprising administering a therapeutically effective amount of an immunotherapeutic agent and a tumor membrane vesicle (TMV), wherein the TMV comprises a lipid membrane, and a B7-1 and/or IL-12 molecule anchored to the lipid membrane, wherein the method for predicting comprises: a) obtaining a blood or serum sample from the subject; b) measuring protein expression levels of biomarkers in the sample, wherein the biomarkers include at least IFN-gamma, TNF-alpha, and IL-2, and wherein an increase in the levels of the biomarkers as compared to a control indicates an increased likelihood the subject will respond therapeutically to the therapy; and c) advising the subject of the increased likelihood the subject will respond therapeutically to the therapy when the relative levels of the biomarkers increase or advising the subject of the decreased likelihood the subject will respond therapeutically to the therapy when the relative levels of the biomarkers do not increase.

The subject can be any mammalian subject, for example a human, dog, cow, horse, mouse, rabbit, etc. In some embodiments, the subject is a primate, particularly a human. The subject can be a male or female of any age, race, creed, ethnicity, socio-economic status, or other general classifiers.

The method predicts the likelihood a subject having a cancer will respond therapeutically to a therapy. Non-limiting examples of cancers include Acute granulocytic leukemia, Acute lymphocytic leukemia, Acute myelogenous leukemia (AML), Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma, Bile duct cancer, Bladder cancer, Bone cancer Bone marrow cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia (CLL), Chronic myelogenous leukemia (CML), Colon cancer, Colorectal cancer, Craniopharyngioma, Cutaneous lymphoma, Cutaneous melanoma, Diffuse astrocytoma, Ductal carcinoma in situ (DCIS), Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors (GIST), Germ cell tumor, Gestational Trophoblastic Disease (GTD), Glioblastoma multiforme (GBM), Glioma, Hairy cell leukemia, Head and neck cancer, Hemangioendothelioma, Hodgkin's lymphoma, Hypopharyngeal cancer, Infiltrating ductal carcinoma (IDC), Infiltrating lobular carcinoma (ILC), Inflammatory breast cancer (IBC), Intestinal Cancer, Intrahepatic bile duct cancer, Invasive/infiltrating breast cancer, Islet cell cancer, Jaw/oral cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Mycosis Fungoides, Myelodysplastic Syndrome, Nasal cavity cancer, Nasopharyngeal cancer, Neck cancer, Neuroblastoma, Neuroendocrine tumors (NETs), Non-Hodgkin's lymphoma, Non-small cell lung cancer (NSCLC), Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system (CNS) lymphoma, Prostate cancer, Rectal cancer, Renal cell carcinoma, Renal pelvis cancer, Rhabdomyosarcoma, Salivary gland cancer, Sarcoma, Sinus cancer, Skin cancer, Small cell lung cancer (SCLC), Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma, Testicular cancer, Throat cancer, Thymoma/thymic carcinoma, Thyroid cancer, Tongue cancer, Tonsil cancer, Transitional cell cancer, Transitional cell cancer, Triple-negative breast cancer, Tubal cancer, Tubular carcinoma, Ureteral cancer, Urethral cancer, Uterine adenocarcinoma, Uterine cancer, Uterine sarcoma, Vaginal cancer, Vulvar cancer, Wilms tumor, Waldenstrom macroglobulinemia, etc., and combinations thereof.

In some embodiments, the cancer comprises breast cancer. In some embodiments, the cancer comprises a triple negative breast cancer. In some embodiments, the triple negative breast cancer comprises a metastatic triple negative breast cancer. In some embodiments, the cancer is prostate cancer.

The immunotherapeutic agent used in the predicting method can be any herein disclosed immunotherapeutic agent. In some embodiments, the immunotherapeutic agent comprises an immune checkpoint inhibitor (ICI). In some embodiments, the immunotherapeutic agent comprises an antibody, particularly an antibody having ICI function. In some embodiments, the immunotherapeutic agent can include one or more of an anti-CTLA4 antibody, an anti-PD1 antibody, an anti-PDL1 antibody, or any combination thereof.

In some embodiments, the anti-CTLA4 antibody can include abatacept, belatacept, ipilimumab, tremelimumab, or any combination thereof. In some embodiments, the anti-CTLA4 antibody is ipilimumab. In some embodiments, the anti-PDL1 antibody can include atezolizumab, durvalumab, avelumab, or any combination thereof. In some embodiments, the anti-PDL1 antibody is atezolizumab (MPDL3280A) (Roche), durvalumab (MEDI4736), avelumab (MS0010718C), or any combination thereof. In some embodiments, the PD-1 inhibitor can include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech). In some embodiments, the anti-PD1 antibody is nivolumab, pembrolizumab, or any combination thereof.

The TMV used in the predicting method can be any herein disclosed TMV. The TMV comprises a lipid membrane and an immunostimulatory agent anchored to the lipid membrane. The immunostimulatory agent used in the predicting method can be any herein disclosed immunostimulatory agent. In some embodiments, the immunostimulatory agent can comprise a full-length polypeptide or, alternatively, can comprise an immunostimulatory portion of full-length immunostimulatory agent.

In some embodiments, the immunostimulatory agent comprises a B7-1, B7-2, or IL-12 molecule. In some embodiments, the immunostimulatory agent comprises a B7-1 or 1-12 molecule. In some embodiments, only a B7-1 molecule is selected. In some embodiments, only a IL-12 molecule is selected. In some embodiments, the TMV comprises both a B7-1 and a IL-12 molecule anchored to the lipid membrane. In some embodiments, the TMV further comprises one or more additional immunostimulatory agents, for instance, B7-2, GM-CSF, and/or IL-2.

In some embodiments, the immunostimulatory agent can be anchored onto the membrane of the TMV through a variety of linkages, such as via lipid palmatic acid, biotin-avidin interaction, or a glycosylphosphatidylinositol (GPI)-anchor.

In some embodiments of the predicting method, the TMV further comprises an antigen molecule anchored to the lipid membrane. The antigen molecule can comprise any herein disclosed antigen molecule. The entire antigen molecule or, alternatively, an antigenic portion of the antigen molecule can be used. In some embodiments, the antigen is a protein, or alternatively, an antigenic fragment of a protein. In some embodiments, the TMV contains an antigen molecule comprising HER-2, PSA, or PAP. Optionally, the antigen molecule is HER-2. In some embodiments, the antigen molecule is the extracellular domain of HER-2 which includes the peptide consisting essentially of amino acids 63-71 of human HER-2 (the “p63-71” peptide) having a sequence of SEQ ID NO:10.

In some embodiments, the antigen molecule may be anchored onto the membrane of the TMV through a variety of linkages, such as via lipid palmatic acid, biotin-avidin interaction, or a glycosylphosphatidylinositol (GPI)-anchor.

In some embodiments, the predicting method further comprises administering an anti-neoplastic agent. In some embodiments, the anti-neoplastic agent can substitute for the immunotherapeutic agent. In some embodiments, the method can include administering the immunotherapeutic agent in combination with an anti-neoplastic agent. The anti-neoplastic agent can be any herein disclosed anti-neoplastic agent.

In some embodiments, the predicting method further comprises administering an adjuvant. The adjuvant can be administered prior to, concurrent with, or subsequent to administration of the TMV and the immunotherapeutic agent. In some embodiments, the adjuvant is GM-CSF, or any biocompatible FDA-approved adjuvant. In some embodiments, the adjuvant comprises IL-2, ICAM1. GM-CSF, flagellin, unmethylated, CpG oligonucleotide, lipopolysaccharides, and lipid A. In some embodiments, the TMV further comprises an adjuvant anchored to the lipid membrane. In some embodiments, the TMV further comprises an adjuvant anchored to the lipid membrane wherein the adjuvant and antigen molecule are not the same molecule.

The predicting method comprises obtaining a blood or serum sample from the subject. The blood or serum sample can be obtained by any suitable, well-known phlebotomy technique. The blood or serum sample is handled, transported, stored, and analyzed under conditions which avoid contaminating or otherwise compromising the integrity of the sample. In an alternative embodiment, the sample from the mammal may be a cancer sample (instead of a blood or serum sample). As used herein, the term “cancer sample” refers to a sample obtained from a mammal suspected of having cancer or known to have cancer, wherein the sample contains cell suspected or known to be cancerous.

The predicting method comprises combining the sample with a therapeutically effective amount of the immunotherapeutic agent and the TMV. In some embodiments, the sample is combined with the immunotherapeutic agent and the TMV in vitro after the sample is obtained. For example, the sample can be combined with the agent in vitro in a flask, tube, microtiter plate, or other laboratory-grade receptacle, and then subjected to the measuring step. In some embodiments, the combining step further comprises agitating, mixing, stirring, vortexing, or other form of homogenizing the combined components. In some embodiments, the sample, immunotherapeutic agent, and TMV are incubated together at ambient conditions for at least 1, at least 5, at least 10, at least 15, at least 30, or at least 60 minutes. In some embodiments, the sample, immunotherapeutic agent, and TMV are incubated together at ambient conditions for at least 1, at least 2, at least 3, at least 4, at least 5, at least 8, at least 12, at least 18, at least 24, at least 36, at least 48, or at least 72 hours.

Alternatively, in some embodiments, a sample is obtained from a subject previously administered the immunotherapeutic agent and the TMV. In such embodiments, the sample can be obtained from the subject essentially immediately after the administration step. Alternatively, the sample can be obtained from the subject at a time after the administration step, for example at least 1 hour, at least 2 hours, at least 3 hours, at least 4 hours, at least 5 hours, at least 10 hours, at least 12 hours, at least 18 hours, or at least 24 hours after the administration step. In some embodiments, the sample can be obtained from the subject at least 1 day, at least 2 days, at least 3 days, at least 4 days, at least 5 days, at least 6 days, at least 7 days, at least 8 days, at least 9 days, at least 10 days, at least 15 days, at least 20 days, at least 25 days, or at least 30 days after the administration step. In some embodiments, the sample is obtained from the subject at about 5 days after the administration step.

The administering step can include any method of introducing the immunotherapeutic agent and TMV into the subject appropriate for the combination therapy formulation. The administering step can include at least one, two, three, four, five, six, seven, eight, nine, or at least ten dosages. The administering step can be performed before the subject exhibits disease symptoms (e.g., prophylactically), or during or after disease symptoms occur. The administering step can be performed prior to, concurrent with, or subsequent to administration of other agents to the subject. The administering step can be performed with or without co-administration of additional agents (e.g., additional immunostimulatory agents, anti-neoplastic agents).

The method can include systemic administration of the immunotherapeutic agent and TMV (e.g., injection into the blood or lymph). Alternatively, the method can include local administration of the immunotherapeutic agent and TMV. For example, the immunotherapeutic agent and TMV can be administered locally to a tumor or an area near a tumor. In some embodiments, the immunotherapeutic agent and TMV are administered to areas of the subject comprising tumors.

In some embodiments, the methods comprise measuring the protein expression level of a set of biomarkers in the sample. The protein expression levels can be measured by any suitable, well-known method to measure protein expression levels. For example, protein expression levels can be measured by a bicinchoninic acid (BCA) assay, Bradford assay, biuret test, absorbance at 280 nm, Lowry method, Coomassie-blue staining, and other suitable methods. In some embodiments, protein expression levels can be measured in a high throughput assay. In some embodiments, protein expression levels can be measured in a multiplex assay which detects two or more, five or more, ten or more, or a plurality of cytokines and/or chemokines. For example, and without limitation, the multiplex assay can be an ELISPOT assay, Fluorispot assay, a Luminex assay, or flow cytometry-based assay. The protein expression levels can be measured within the sample as obtained from the subject (e.g., in whole blood or serum). Alternatively, cells (e.g., immune cells, and specifically T-cells) can be isolated from the sample and protein expression levels can be measured using those immune cells from the sample. In some embodiments, the sample can be further modified prior to measuring protein expression levels. For example, immune cells from the sample can be lysed and protein expression determined using total cell lysate.

The set of biomarkers measured in the sample include at least IFN-γ, TNF-α, and IL-2. In some embodiments, the set of biomarkers further comprises IL-12 or IL-18, or both IL-12 and IL-18. In some embodiments, the set of biomarkers further comprises IL-22 or IL-23, or both IL-22 and IL-23. In some embodiments, the set of biomarkers further comprises at least one, at least two, or at least three biomarkers selected from the group consisting of IL-12, IL-18, IL-22, and IL-23. In some embodiments, the set of biomarkers further comprises each of IL-12, IL-18, IL-22, and 1-23.

In some embodiments, the set of biomarkers further comprises, for example, IL-103, IL6, IL-12, IL-15, IL-18, IP-10, GM-CSF, IL-4, IL-5, IL-13, IL-31, IL-17A, IL-22, 1-23, IL-27, IL-28, ENA-78, CXCL-1, MIP-1 β, LIF, or any combination thereof. In some embodiments, the set of biomarkers measured in the sample include at least IFN-γ, TNF-α, and IL-2 and at least one, at least two, at least three, at least four, at least five, at least six, at least seven, at least eight, at least nine, or at least ten, or more additional biomarkers selected from the group consisting of IL-1β, IL6, IL-12, I-15, IL-18, IP-10, GM-CSF, IL-4, IL-5, IL-13, IL-31, IL-17A, IL-22, IL-23, IL-27, 1-28, ENA-78, CXCL-1, MIP-1 β and LIF.

The levels of biomarkers are compared to a control. The control can be a biological sample. Alternatively, a collection of values used as a standard applied to one or more subjects (e.g., a general number or average that is known and not identified in the method using a sample). In some embodiments, the control comprises a blood or serum sample obtained from the subject prior to the administration step (e.g., a baseline sample).

When compared to a control, an increase in the level of the biomarkers indicates an increased likelihood the subject will respond therapeutically to the therapy. In some embodiments, the amount of increase in the level of the biomarkers which indicate an increased likelihood the subject will respond therapeutically to the therapy can be any amount which is statistically significant. In some embodiments, the amount of increase which indicates an increased likelihood of therapeutic response can be at least 1%, at least 2%, at least 3%, at least 5%, at least 7%, at least 10%, at least 15%, at least 20%, at least 25%, at least 30%, at least 40%, at least 50%, at least 100%, or more as compared to a control.

Because the methods include measuring the protein expression level of a set of biomarkers, it is possible that the level of some biomarkers will increase as compared to a control, while the level of other biomarkers will not increase (or alternatively, decrease) as compared to a control. In some embodiments, an increase in the level of at least one biomarker indicates an increased likelihood the subject will respond therapeutically to the therapy. In some embodiments, an increase in the level of at least two or at least three biomarkers indicates an increased likelihood the subject will respond therapeutically to the therapy. In some embodiments, an increase in the level of at least four, at least five, at least six, at least seven, at least eight, at least nine, or at least ten or more biomarkers indicates an increased likelihood the subject will respond therapeutically to the therapy.

In some embodiments, the method comprises advising the subject of the increased likelihood the subject will respond therapeutically to the therapy when the relative levels of the biomarkers increase or advising the subject of the decreased likelihood the subject will respond therapeutically to the therapy when the relative level of said biomarkers does not increase.

In some embodiments in which the subject is advised of the increased likelihood the subject will respond therapeutically to the therapy, the method further comprises treating the subject with an effective amount of the therapy.

Also disclosed herein is a method for measuring an anti-tumor immune response (i.e. tracking a patient's response) in a mammal, the method comprising administering a tumor membrane vesicle (TMV) and an immunotherapeutic agent; measuring in a blood or serum sample of the mammal an amount of a set of biomarkers comprising IFN-gamma, TNF-alpha, and IL-2, and predicting the likelihood a mammal will respond therapeutically to a therapy based on an increased amount of the biomarkers compared to a control.

In some embodiments, the methods disclosed herein further comprise detecting an antigen molecule. In some embodiments, the antigen molecule comprises HER-2. In some embodiments, the antigen molecule comprises PSA. In some embodiments, the antigen molecule comprises PAP. In some embodiments, the antigen molecule is selected from HER-2, MKI67, prostatic acid phosphatase (PAP), prostate-specific antigen (PSA), prostate-specific membrane antigen, early prostate cancer antigen, early prostate cancer antigen-2 (EPCA-2), BCL-2, MAGE antigens such as CT7, MAGE-A3 and MAGE-A4, ER 5, G-protein coupled estrogen receptor 1, CA15-3, CA19-9, CA 72-4, CA-125, carcinoembryonic antigen, CD20, CD31, CD34, PTPRC (CD45), CD99, CD 117, melanoma-associated antigen (TA-90), peripheral myelin protein 22 (PMP22), epithelial membrane proteins (EMP-1, -2, and -3), HMB-45 antigen, MART-1 (Melan-A), S100A1, S100B and gp 100:209-217(210M), MUC-1, mucin antigens TF, Tn, STn, glycolipid globo H antigen. Typically, the antigen is the human form. The antigen molecule can be detected by any suitable method known in the art.

In one embodiment, the methods disclosed herein further comprise determining the bioactivity of B7-1 and/or IL-12 using a reporter cell selected from Jurkat E6.1, NK-92, NK-92MI, or HEK-Blue IL-12 cells.

### Methods for Testing

Further disclosed herein are methods of testing the TMV compositions disclosed herein. Included herein is a method of testing a tumor membrane vesicle (TMV) composition comprising: combining the TMV composition with one or more human NK-92 cells in a sample; and determining the amount of IFN-gamma in the sample after the combination; wherein an increase in the amount of IFN-gamma in the sample following the combination indicates an active TMV composition. An active TMV composition is defined herein as one that is capable of initiating an immune response, or more particularly, a T cell response. The amount of IFN-gamma in the sample is determined after the TMV composition is combined with one or more human NK-92 cells. The amount of IFN-gamma can be determined following an approximately 12 hour, an approximately 24 hour, an approximately 36 hour, an approximately 48 hour, or an approximately 60 hour combination or co-culture. In some embodiments, the amount of IFN-gamma is determined between an approximately 48 hour combination or co-culture.

### Kits

Also disclosed herein is a kit comprising an IFN-γ detecting agent, a TNF-α detecting agent, and an IL-2 detecting agent. In some embodiments, the kit further comprises at least one of an IL-12 detecting agent, an IL-18 detecting agent, an IL-22 detecting agent, an IL-23 detecting agent, or any combination thereof. In some embodiments, the kit further comprises at least one of an IL-10 detecting agent, an IL6 detecting agent, an IL-12 detecting agent, an IL-15 detecting agent, an IL-18 detecting agent, an IP-10 detecting agent, a GM-CSF detecting agent, an IL-4 detecting agent, an IL-5 detecting agent, an 1-13 detecting agent, an 1-31 detecting agent, an IL-17A detecting agent, an IL-22 detecting agent, an IL-23 detecting agent, an IL-27 detecting agent, an IL-28 detecting agent, an ENA-78 detecting agent, a CXCL-1 detecting agent, a MIP-1 β detecting agent, a LIF detecting agent, or any combination thereof.

In some embodiments, the detecting agent is an antibody or fragment thereof which specifically binds the cytokine or chemokine for which the agent detects. In some embodiments, the antibody or fragment thereof is conjugated to detection compound (e.g., a fluorescent or enzymatic reporter).

In some embodiments, the kit further comprises reagents for use in detecting one or more biomarkers (e.g., a cytokine or chemokine) when a sample is combined with a detecting agent. In some embodiments, the kit further comprises a receptacle (e.g., tubes, microtiter plate) for analyzing each biomarker in a separate detection reaction.

The kit is suitable for analyzing a sample to predict the likelihood a human subject having a cancer will respond therapeutically to a therapy that comprises administering a therapeutically effective amount of an immunotherapeutic agent and a tumor membrane vesicle (TMV), wherein the TMV comprises a lipid membrane, a B7-1 or IL-12 molecule anchored to the lipid membrane, and an antigen molecule anchored to the lipid membrane.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. Tumor Membrane Vesicle (TMV) Immunotherapy and Immune Checkpoint Inhibitor Combination Therapies for Triple Negative Breast Cancer

### Methods

Production and Purification of GPI-Proteins.

GPI-mB7-1 and GPI-mIL-12 were expressed in CHO-K1 cells and immunoaffinity purified as previously described (Patel et al, 2015).

Mice.

6-8 week old female BALB/cJ mice were purchased from Jackson Laboratories (Bar Harbor, Me.) and maintained in accordance with Emory University IACUC approved institutional guidelines and protocols.

Antibodies.

Purified hamster anti-mouse CD80 (Clone 16-10A1 or 1G10), rat-anti-mouse CD80 (Clone 1G10) and rat anti-mouse IL-12 p40 (Clone C17.8) used for affinity chromatography were purchased from BioXCell (West Lebanon, N.H.). Fluorochrome-conjugated anti-mouse monoclonal antibodies specific for murine CD80 (Clone 16-10A1) was purchased from BD Biosciences (San Diego, Calif.) and IL-12 p40 (Clone C17.8) was purchased from eBioscience (San Diego, Calif.) and used to assess GPI-mB7-1 and GPI-mIL-12 incorporation into 4T1 TMVs.

Anti-mouse CTLA-4 antibody (Clone 9D9) and anti-mouse PD-L1 antibody (Clone 10F.9G2) were purchased from BioXCell. Anti-mouse CD4 (Clone GK1.5) and CD8 (YTS 169.4) antibodies used for cell depletion (500 μg dose given i.p. once in 200 μl PBS) were also purchased from BioXcell.

Cell Lines.

4T1 cells were purchased from ATCC (Manassas, Va.) and maintained in DMEM (Corning, Manassas, Va.) containing 10% FBS (Hyclone, Logan, Utah), L-Glutamine, and Penicillin/Streptomycin. CHO-K1 cells were obtained from ATCC and grown in RPMI (Corning) containing 10% CCS (Hyclone), L-Glutamine, HEPES, and Penicillin/Streptomycin.

Tumor Challenge Studies.

4T1 cells (2×10⁴) were suspended in 100 μl of phosphate-buffered saline (PBS) and injected into the second mammary fat pad on the right. Tumors were resected on day 9-10 upon reaching a palpable 3-4 mm in size. Mice were then treated with immunotherapy and followed to assess survival or metastasis. Mice were euthanized when body weight decreased by >10% or when they became moribund.

Immunotherapy Studies.

For metastasis and survival studies, 4T1 membrane-based immunotherapy (100 μg TMV containing 2.5 μg of GPI-protein per 100 μg TMV) was given s.c. to some groups 2 days after surgery and a booster dose given 9 days after surgery. 4T1 TMV were prepared and incorporated with GPI-proteins as previously described. See McHugh et. al., PNAS, 92:8059-63 (1995). Briefly, tumors were grown s.c. in the hind flanks and excised upon reaching 10 mm in diameter and frozen at −80° C. Tumors were then minced and homogenized using a disposable Omni tip homogenizer (Omni International, Kennesaw, Ga.) and centrifuged over a 41% sucrose gradient at 100,000×g. TMV were collected from the interface, washed, and resuspended in PBS. TMV concentration was then determined using a micro BCA assay (Thermo Scientific, Rockford, Ill.). TMV were then incorporated with GPI-proteins at 2.5 μg/100 μg TMV for 4 h at 37° C. with gentle rotation, centrifuged, and resuspended in PBS prior to injection at 1 mg/ml final concentration. Incorporation of GPI-mB7-1 (anti-mouse CD80-APC, Clone 16-10A1) and GPImIL-12 (anti-mouse IL-12 p40-PE, Clone C17.8) was evaluated using flow cytometry (data not shown). For immune response studies, mice were given 3 injections of membrane-based immunotherapy spaced at 2-week intervals. Immune responses were then evaluated 4 weeks after the final dose.

Anti-CTLA-4 mAb (Clone 9D9) was given i.p. 1 day following TMV immunotherapy and subsequently every 3 days for a total of 4 doses: dose 1 (200 μg), 2 (100 μg), 3 (100 μg), and 4 (100 μg) in 200 μl PBS. This dosing schedule was adapted from a published study. See Waitz et. al., Canc. Res. 72:430-9 (2012). For immune response studies, anti-CTLA-4 mAb was only given twice after each TMV immunotherapy: on dl (200 μg) and d4 (100 μg) post-treatment for a total of 6 injections.

Anti-PD-L1 mAb (Clone 10F.9G2) was given i.p. 1 day following TMV immunotherapy and subsequently every 3 days for a total of 4 doses: dose 1 (200 μg), 2 (200 μg), 3 (200 μg), and 4 (200 μg) in 200 μl PBS.

Cyclophosphamide (Sigma, St. Louis, Mo.) was administered once i.p. at 50 mg/Kg in 200 PBS on day 1 post TMV immunotherapy. See Zhao et. al., Canc. Res. 70:4850-58 (2010).

Clonogenic Assay.

For the clonogenic assay, the membrane based immunotherapy and anti-CTLA-4 mAb therapy were given as described above. A clonogenic assay was performed as previously described. See Franken et. al., Nat. Prot. 1:2315-19 (2006). Briefly, mice were sacrificed on day 28-35 post tumor challenge, lungs homogenized and processed to a single cell suspension using collagenase type IV (Sigma) in a 1 hour incubation at 37° C., and then passed through a 70 μM cell strainer. Cells were washed and suspended in DMEM with 10% FBS (Hyclone) containing 6-thioguanine (Sigma). Serial dilutions were made and cultures were grown for 7 days at 37° C., 5% CO₂. 4T1 cells are resistant to 6-thioguanine, but normal lung cells are sensitive and fail to survive, leading to the formation of distinct 4T1 colonies. Colonies were visualized using 0.5% crystal violet and counted.

Statistical and Flow Cytometry Analysis.

A log-rank (Mankel-Cox) test was used to evaluate Kaplan-Meier survival curves. Student's t-test (unpaired, 2-tailed) was used to compare experimental groups. P values <0.05 were deemed statistically significant (*p<0.05, **p<0.01). Graphpad Prism 6 software was used to analyze data. FloJo version 9.7.6 software was used for flow cytometry data analysis (TreeStar, Ashland, Oreg.).

### Results

Mice Treated with Combination TNBC Tumor Membrane-Based Immunotherapy and Anti-CTLA-4 Antibody Demonstrated Increased Survival in a TNBC Tumor Resection Model.

To more closely mimic the clinical setting, the immunotherapy was evaluated using a TNBC tumor resection model, since patients would begin receiving immunotherapy as a post-operative treatment to reduce metastasis and improve survival. Treatment efficacy was evaluated using the 4T1 orthotopic breast cancer model. The tumor challenge, resection, and immunotherapy protocol is described in the methods section. Primary tumors were resected on day 10, by which time a palpable tumor was observed and spontaneous metastasis had occurred. See Pulaski et. al., Curr. Prot. Imm., Ch. 20, Unit 20.2 (2001).

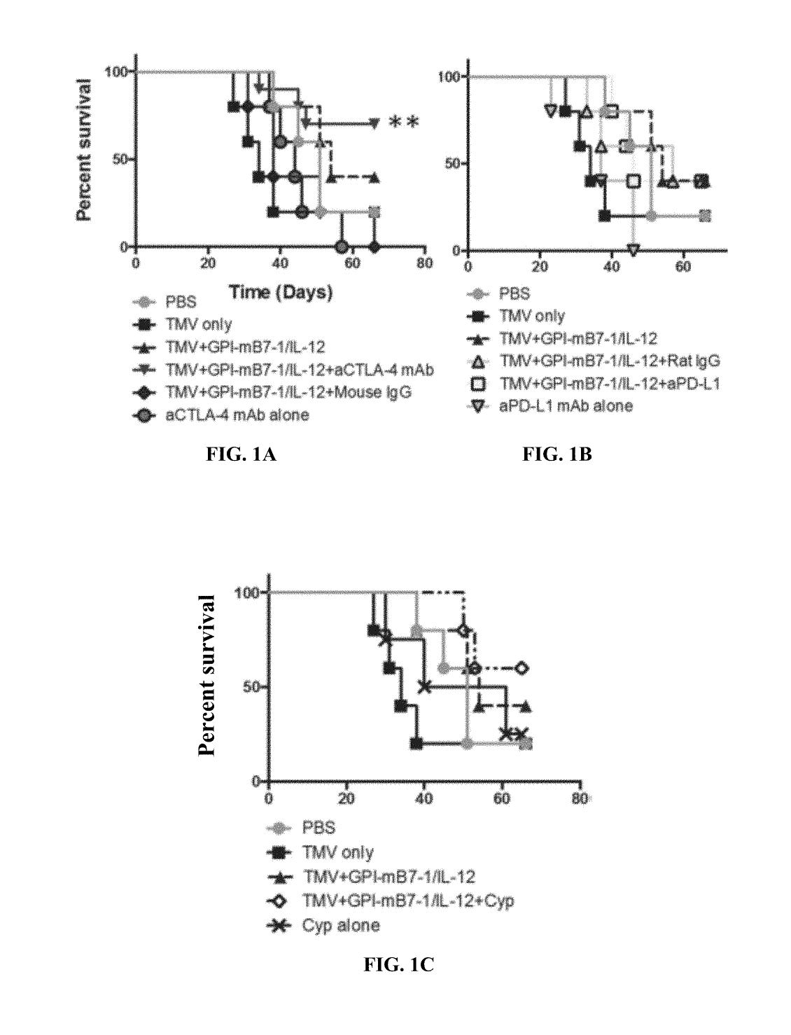

The majority of mice (80%) in the control groups died by day 40. However, 70% (7/10) mice survived without signs of disease up to day 66 in the combination therapy group (FIG. 1A). The combination therapy group was treated with (1) tumor membrane vesicles (TMV) formed from cellular membranes of triple negative breast cancer (TNBC) model 4T1 cells (hereinafter, 4TI TMV), wherein the 4TI TMVs contained both GPI-anchored mB7-1 and GPI-anchored IL-12 (hereinafter, GPI-mB7-1/IL-12), and (2) an anti-CTLA-4 antibody (hereinafter, aCTLA-4 mAb). Interestingly, anti-CTLA-4 mAb therapy alone provided no significant increase in survival, as 80% of mice died before day 48 and 100% by day 60 (FIG. 1A). A log-rank analysis (Mankel-Cox test) demonstrated that a significant increase in survival was observed in the combination therapy group receiving TMV immunotherapy and anti-CTLA-4 mAb treatment in comparison to anti-CTLA-4 mAb therapy alone or TMV-based vaccine and mouse IgG (control for anti-CTLA-4 mAb) (FIG. 1A).

A number of other combination therapies were also evaluated in conjunction with the immunotherapy approach. Strikingly, combination therapy with anti-PD-L1 mAb did not increase survival (FIG. 1B), despite high level expression of PD-1 on 4T1 tumor infiltrating CD8+ T cells and upregulated expression of PD-L1 on 4T1 cells following exposure to IFN-γ in vitro (data not shown). In addition, combination therapy with a single low-dose cyclophosphamide (Cyp) treatment increased survival compared to the untreated group on 40^{th} day, but showed no difference on day 60 (FIG. 1C). Failure of both the TMV-Cyp combination therapy and, in particular, the TMV-anti-PD-L1 mAb combination therapy to produce therapeutically acceptable results underscores the unpredictability (and frequent failure) of candidate treatments for TNBC which can otherwise produce therapeutic results in other cancers, even other breast cancers.

Reduction in Lung Metastasis in Mice Treated with a TNBC Membrane-Based Immunotherapy Prior to Tumor Challenge and an Anti-CTLA-4 Antibody Therapy Post-Challenge.

The primary cause of death in the 4T1 model is pulmonary metastasis. See Pulaski et. al., Curr. Prot. Imm., Ch. 20, Unit 20.2 (2001). Therefore, it is likely that a reduction in metastasis to the lungs may be a contributing factor to the increased survival observed in mice co-administered with the membrane-based immunotherapy and anti-CTLA-4 antibody therapy. To address this, Balb/c mice were immunized prior to orthotopic tumor implantation. Mice were given immunotherapy 21 days and 7 days before 4T1 tumor challenge. Anti-CTLA-4 or anti-PD-L1 antibody therapy was administered as described in the methods section.

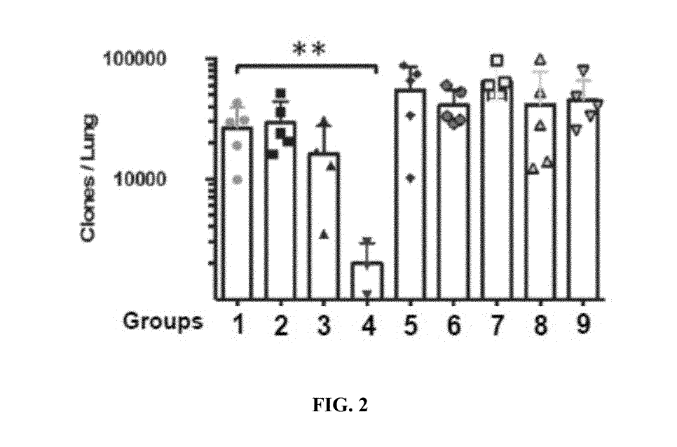

After 27-28 days, the mice were sacrificed and a clonogenic assay was performed on lung tissue to assess the extent of metastasis (FIG. 2). Interestingly, only the group receiving both TMV immunotherapy and anti-CTLA-4 antibody treatment demonstrated a significant reduction in the number of colonies present in lung tissue. Mice receiving combination therapy with the membrane-based immunotherapy and anti-CTLA-4 mAb therapy (FIG. 2, group 4) showed a reduction of approximately 90% in the number of metastatic cells detected (20,000 colonies in the control PBS group versus 2000 colonies in the combination therapy group). No reduction in metastasis was detected in mice receiving combination therapy with anti-PD-L1 antibody (FIG. 2, group 7).

Reduction in Metastasis to the Lung in Mice Given a TNBC Membrane-Based Immunotherapy in Combination with Anti-CTLA-4 Antibody Post Tumor Challenge and Surgical Resection.

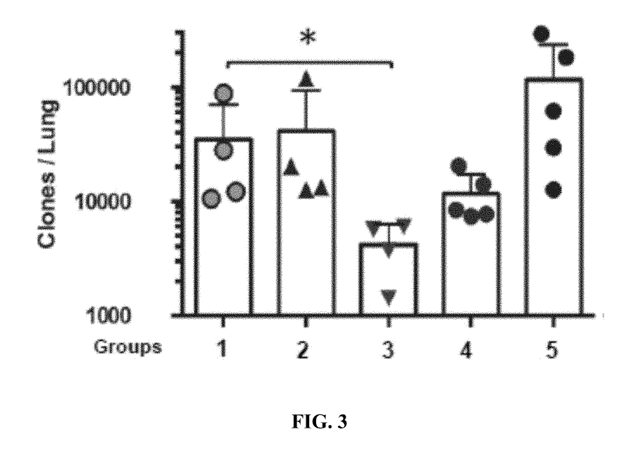

To more closely recapitulate the clinical setting, it was tested whether the observed metastasis reduction in a prophylactic immunization setting would be observed following a therapeutic challenge. To accomplish this, BALB/c mice were challenged orthotopically with 4T1 cells, and then tumor was resected as previously described. Mice were given immunotherapy 2 days and 9 days after 4T1 tumor resection. Anti-CTLA-4 antibody therapy was administered post resection, as described in the methods section. After 35-36 days, the mice were sacrificed and a clonogenic assay was performed on lung tissue to assess the extent of metastasis (FIG. 3). Again, only the group receiving both immunotherapy and anti-CTLA-4 antibody treatment (FIG. 3, group 3) demonstrated a significant reduction in the number of colonies present in lung tissue. Mice receiving combination therapy with the membrane-based immunotherapy and anti-CTLA-4 mAb therapy showed a reduction of approximately eight-fold in the number of metastatic cells detected (approximately 20,000 colonies in the control PBS group versus approximately 2500 colonies in the combination therapy group). Of note, the anti-CTLA-4 treatment group appears to show a reduction in metastasis. However, results were not significant when compared to the PBS control group.

CD8 T Cells are Required to Control Metastasis in the 4T1 Triple Negative Breast Cancer Model.

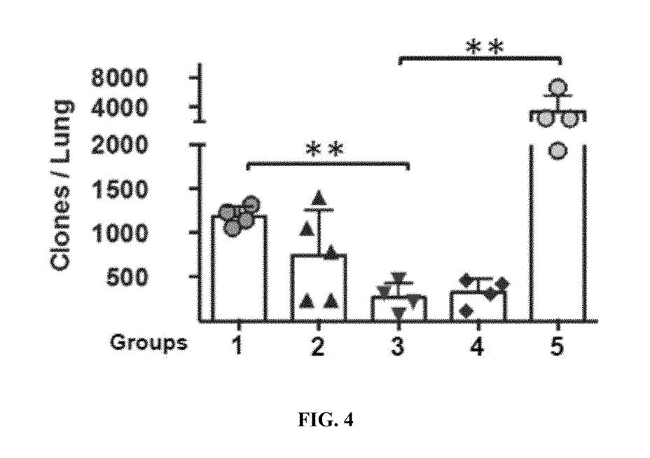

Cell depletion experiments were carried out to determine which cell population was responsible for protection against 4T1 metastasis. Anti-mouse CD4 (Clone GK1.5) and CD8 (YTS 169.4) antibodies were used for cell depletion. Depletion of CD4 T cells (FIG. 4, group 4) prior to tumor challenge yielded no difference in control of metastasis. However, depletion of CD8 T cells (FIG. 4, group 5) resulted in loss of control of 4T1 metastasis, with a 10-fold increase in comparison to mice treated with immunotherapy and anti-CTLA-4 antibody. Interestingly, metastasis in the CD8 T cell-depleted mice was approximately 4-fold higher than the control mice, suggesting that non-tumor specific CD8 T cells also play a role in control of 4T1 metastasis or base line level of priming induced by tumor in the absence of vaccination may be involved in partially controlling the metastatic growth.

TMVs Harboring GPI-Anchored IL-12 Exhibit No Liver Toxicity.

The immunostimulatory agent (ISM) IL-12 administered in its soluble form is associated with chronic liver toxicity. To determine whether TMVs having GPI-anchored 1-12 also cause toxicity, a complete liver toxicity profile was performed. For purposes of this experiment, dosage levels of total TMVs were defined as a high dose (300 μg), a standard dose (100 μg), and a low dose (50 μg). Mouse groups were administered PBS alone, anti-CTLA-4 mAb alone, high, standard, and low doses of TMV+GPI-B7-1+GPI-IL-12, as well as high, standard, and low doses of TMV+GPI-B7-1+GPI-IL-12+anti-CTLA-4 mAb combination therapy. Serum was drawn from each group receiving treatments, and multiple serum draws were collected and pooled for each group for toxicity analysis. The following clinical chemistry tests were performed by Charles River Laboratories: Alanine aminotransferase (ALT), Albumin (ALB), Alkaline phosphatase (ALK), Aspartate aminotransferase (AST), Total Bilirubin (TBIL), and Total Protein (TP).

Table 1 shows the full liver toxicity profile data. Of particular interest is that there appears to be no difference in any performed test between all groups, suggesting that no liver toxicity is observed under these conditions. Previous studies with soluble IL-12 tend to induce toxicity in such a liver profile, especially in the ALT and AST tests. These data show that membrane anchoring of IL-12 mitigates systemic toxicity issues. For convenience, the immunostimulatory agents B7-1 and IL-12 are collectively referred to as “ISM” in Table 1. Thus, the TMVs contain both GPI-B7-1 and GPI-IL12. The dosage levels of TMVs (50, 100, and 300 μg) refer to amounts of total TMVs.

TABLE 1
Liver toxicity profiles.

| Groups for Liver | ALK | ALT | AST | TBIL | ALB | TP |
|---|---|---|---|---|---|---|
| Toxicity Profile | (U/L) | (U/L) | (U/L) | (mg/dL) | (g/dL) | (g/dL) |
| PBS | 111 | 18 | 49 | 0.2 | 3.1 | 4.8 |
| anti-CTLA-4 mAb | 100 | 21 | 56 | 0.2 | 3.3 | 5.0 |
| alone | | | | | | |
| TMV + GPI-ISM | 106 | 21 | 48 | 0.2 | 3.1 | 4.9 |
| (300 μg) | | | | | | |
| TMV + GPI-ISM | 108 | 18 | 50 | 0.2 | 3.1 | 4.9 |
| (300 μg) + | | | | | | |
| anti-CTLA-4 mAb | | | | | | |
| TMV + GPI-ISM | 100 | 20 | 48 | 0.2 | 3.1 | 5.2 |
| (100 μg) | | | | | | |
| TMV + GPI-ISM | 97 | 23 | 49 | 0.2 | 3.3 | 5.2 |
| (100 μg) + | | | | | | |
| anti-CTLA-4 mAb | | | | | | |
| TMV + GPI-ISM | 103 | 26 | 75 | 0.2 | 3.2 | 5.0 |
| (50 μg) | | | | | | |
| TMV + GPI-ISM | 97 | 23 | 55 | 0.2 | 3.2 | 5.0 |
| (50 μg) + | | | | | | |
| anti-CTLA-4 mAb | | | | | | |

### CONCLUSIONS

Results disclosed herein show that a combination of tumor membrane-based immunotherapy along with anti-CTLA-4 mAb therapy work together to suppress metastasis, stimulate CD8 T cell immunity, and enhance overall survival in the highly aggressive, poorly immunogenic 4T1 murine breast cancer model. In addition, protection appears to depend on CD8 T cell immunity, not anti-tumor antibody responses. Further, administration of TMVs containing GPI-anchored 1-12 causes no liver toxicity. This combinatorial approach is a promising treatment option for TNBC patients that critically need additional approaches to help combat their disease and prevent relapse.

### Example 2. Biomarkers of Tumor Membrane Vesicle (TMV)-Based Immunotherapy

Immune dysfunction is associated with tumor progression and metastasis in cancer patients. Tumors evade host immune system by numerous mechanisms such as suppression, anergy or deletion of effector T cells. Activating the host immune system against tumor associated antigens by active immunization with tumor antigens, and releasing the brakes on the immune system by administering antibodies against immune checkpoint inhibitors can lead to elimination of tumors. This example describes personalized immunotherapy vaccines consisting of tumor membrane vesicles modified with GPI-anchored immunostimulatory agents IL-12 and B7-1. Immune activity of TMV-based vaccine in combination with immune checkpoint inhibitor antibodies (for example, anti-CTLA-4 and/or anti-PD-1 antibodies) is measured using in vitro biological assays (ELISPOT, Cytokine secretion etc.) and immune response biomarker assays (serum cytokines) as outlined in this example below. This example also describes TMV vaccine preparation and modification with GPI-anchored immunostimulatory agents B7-1, IL-12 etc; TMV-based vaccine activity assay using reporter cell lines; immune response to TMV-based vaccine in pre-clinical rodent model using ELISPOT, serum cytokines and chemokines biomarkers; and antitumor response in tumor bearing mice induced by TMV-based vaccine in combination with immune checkpoint blockade therapy antibodies and standard of care chemotherapy drugs (survival and clonogenic assays).

### Cytokine ELISPOT Assay (IL-2, IFN-γ, TNF-α)

Methods.

In mice, spleen cells were isolated on day 28-35 after the final immunization and a single cell suspension made by mincing spleens with a scissor and then passing through a cell strainer. Red blood cells (RBC) were lysed with RBC lysis buffer (Sigma) and spleen cells suspended in RPMI-1640 containing 10% FBS (Hyclone), L-Glutamine, 50 μM 2-mercaptoethanol, and 10 mM HEPES. 0.5×10⁶ cells were plated into each well of a nitrocellulose 96-well ELISPOT plate (Millipore, Billerica, Mass.) previously coated with rat anti-mouse IFN-γ antibody (Clone R4-6A2, BD Biosciences). Cells were stimulated with 1 μM HER-2 p63-71 peptide or mitomycin C (Sigma) treated (100 μg/ml for 2 hours) 4T1 cells at a 20:1 ratio (5×10⁵ spleen cells: 25,000 4T1 cells) for 48 hours at 37° C., 5% CO₂. After washing off the cells, biotin rat anti-mouse IFN-γ (Clone XMG1.2, BD Biosciences) was added and incubated at 4° C. overnight. The following day streptavidin HRP (BD Biosciences) was added and spot-forming units (SFU) were revealed using 3-amino-9-ethyl-carbazole substrate (AEC) (Sigma). Plates were dried, and the total number of spots were counted using a dissecting microscope. This assay was adapted from a published study. See Chen et. al., Mol. Ther., 15:2194-202 (2007). In addition, this assay could be adapted to blood cells (PBMC) and patient-specific TMV previously modified with GPI-ISMs (@10-40 μg/ml) is used as the antigen source. For patients expressing high levels of HER-2, the HER-2 p63-71 peptide is used at a concentration of 1-10 μM. Other key cytokines are added to the testing panel, including IL-2 and TNF-alpha. The matching anti-human cytokine antibody pairs for these assays are available from BD Biosciences.

Results.

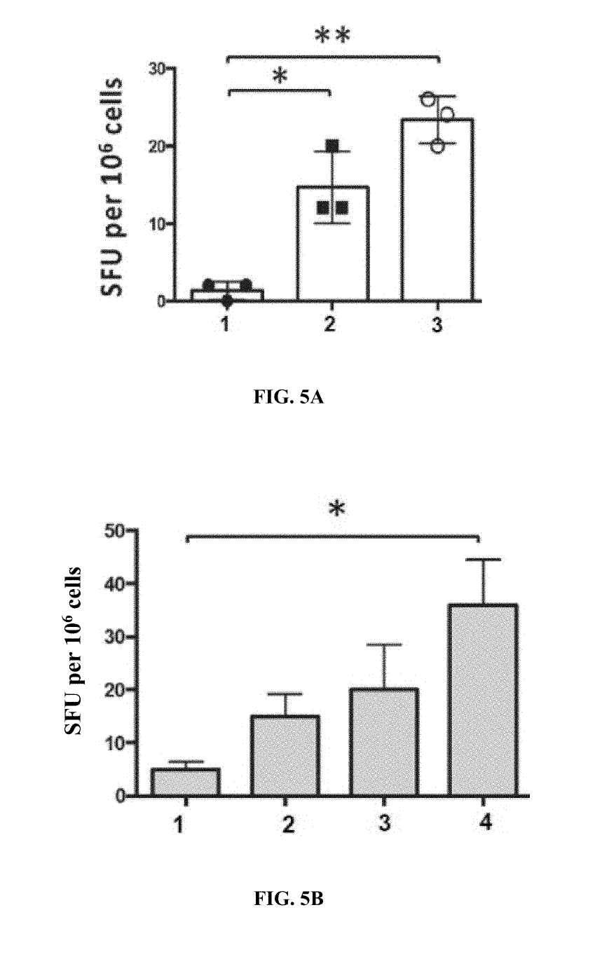

ELISPOT assays were used to monitor anti-tumor immune responses in the blood following TMV-based immunotherapy in the clinic. CD8 T cells are a primary immune cell contributor to control and clearance of metastatic tumor colonies. Unfortunately, the MHC Class I-restricted peptide epitopes have not yet been defined in the 4T1 system, making it difficult to track antigen-specific CD8 T cell responses. In an effort to follow the magnitude of CD8 T cell response against a known CD8 T cell epitope delivered via membrane-based immunotherapy, a known tumor antigen (human HER-2 extracellular domain attached to GPI anchor) was inserted into TMVs prepared from 4T1 tumor tissue. Since the human HER-2 extracellular domain contains an H-2K^{D} restricted epitope (p63-71), this protein allows for the inclusion and monitoring of the CD8 T cell response to a model breast cancer antigen. See Nagata et. al., J. Immunol. 159(3): 1336-43 (1997). Mice were immunized as previously described, except only one boost was given. After 10 days the spleen was removed and an IFN-γ ELISPOT assay was performed to assess the magnitude of the CD8 T cell response to GPI-anchored human HER-2 Extracellular Domain containing the H-2K^{D} restricted epitope p63-71 (hereinafter “hHER-2ED”). Although a significant hHER-2ED specific response was clearly observed in mice that were given TMV+GPI-hHER-2ED/ISM alone (FIG. 5A, group 2), addition of anti CTLA-4 mAb enhanced the magnitude of the peptide-specific response from 15 SFU/10⁶ cells to 22 SFU/10⁶ cells (FIG. 5A, group 3).

In addition, the immune response against 4T1 cells was also evaluated. In brief, spleen cells were incubated with mitomycin C treated 4T1 cells for 48 hours and the number of IFN-γ secreting cells was evaluated using an ELISPOT assay as above. The IFN-γ response against 4T1 cells was augmented when a combination therapy approach with anti-CTLA-4 antibody was employed, increasing from approximately 20 SFU/10⁶ cells to 35 SFU/10⁶ cells (FIG. 5B), thereby showing that combination therapy significantly enhances anti-4T1 tumor-specific immunity when compared to mice treated with TMV only. Serum antibody responses against Chinese hamster ovary (CHO) cells expressing human hHER-2ED (in 4T07-human HER-2 TMV immunized mice). The 4T07 cell line is a non-metastatic murine breast cancer cell line. yielded no significant difference in serum antibody against HER-2, since all immunized groups generated strong anti-HER-2 antibody titers regardless of combination with anti-CTLA-4 mAb (data not shown). However, when serum antibody responses against 4T1 cells was evaluated, no significant anti-4T1 antibody titers were detectable following immunotherapy, even when TMV vaccination was combined with anti-CTLA-4 mAb (data not shown).

### Luminex Assay

Methods.

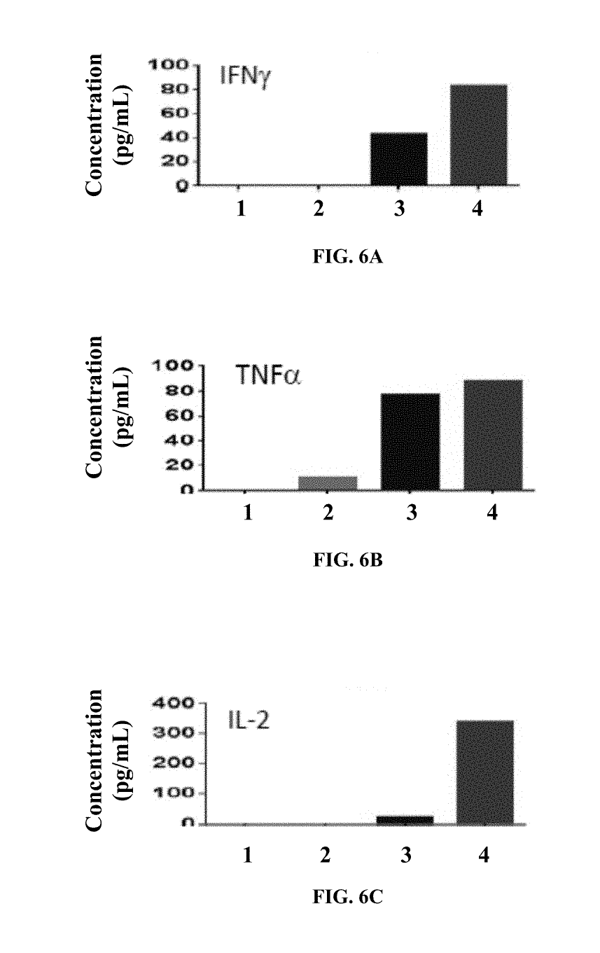

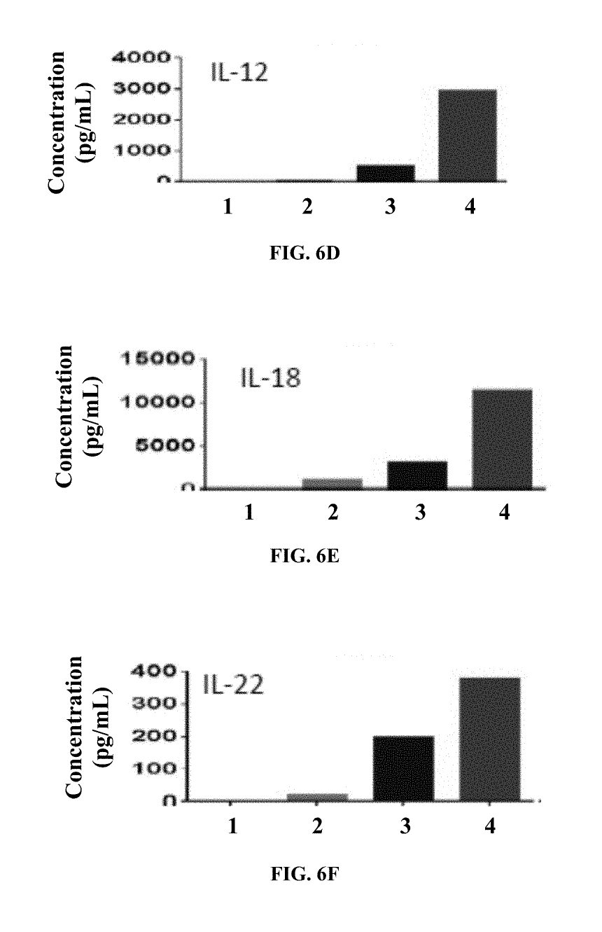

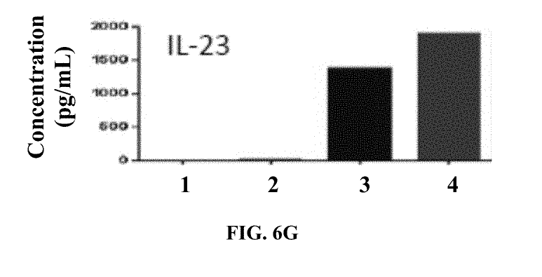

Mice were bled on day 5 after the final immunization and serum was collected. Samples were pooled from each group and an e-Bioscience 36-plex Luminex assay was performed by Charles River Laboratories (Wilmington, Mass.). Results are depicted in FIG. 6(A-G). Samples were run in duplicate and cytokine/chemokine amounts were extrapolated using a standard curve for each protein. Cytokines that were detected at high levels in the serum included IL-2, TNF-α, IFN-γ, IL-12, IL-18, IL-22, and IL-23

Results.

When serum was taken from mice on day 5 after the final immunization, mice were immunized with PBS (group 1), anti-CTLA-4 antibody (group 2), 4T1 TMV-GPI-ISM (group 3), or 4T1 TMV-GPI-ISM+anti-CTLA-4 antibody (group 4). 100 μg 4T1 TMV-GPI-ISM was used for three injections at a fourteen-day interval. Anti-CTLA-4 antibody was administered i.p. on days 3 and 6 after TMV vaccination, and pooled serum analyzed by a 36-plex immune array (eBioscience Luminex), mice receiving the combination therapy had markedly increased levels of several key inflammatory cytokines and chemokines in comparison to anti-CTLA-4 mAb alone of TMV immunotherapy alone (FIG. 6A-G). Almost no measurable response was observed in mice receiving PBS or anti-CTLA-4 mAb alone. Specifically, combination therapy increased serum concentrations in comparison to TMV immunotherapy alone from: IL-1β, 34 to 100 pg/ml; IL-6, 0 to 165 pg/ml; IL-12, 502 to 2956 pg/ml; IL-28, 138 to 289 pg/ml; IL-2, 24 to 337 pg/ml; IFN-γ, 43 to 83 pg/ml; TNF-α, 77 to 88 pg/ml; IP-10, 82 to 213 pg/ml; IL-4, 109 to 395 pg/ml; IL-5, 50 to 137 pg/ml; IL-13, 23 to 106 pg/ml; IL-31; 82 to 725 pg/ml; IL-17a, 6 to 28 pg/ml; IL-22. 198 to 379 pg/ml; IL-23, 1379 to 1891 pg/ml; IL-27, 110 to 212 pg/ml; IL-15, 16 to 54 pg/ml; IL-18, 3074 to 11434 pg/ml; and GM-CSF, 107 to 526 pg/ml. In addition, a substantial increase in serum concentration in the combination therapy group versus TMV immunotherapy alone was also detected for certain chemokines (ENA-78, 49 to 336 pg/ml; CXCL-1 119 to 672 pg/ml; MIP-2, 71 to 231 pg/ml; MIP-1b, 46 to 196 pg/ml; and LIF, 36 to 155 pg/ml). No marked changes were observed for IL-1α, G-CSF, M-CSF, IFN-α, IL-3, IL-10, IL-9, Eotaxin, MCP-3, MIP-1α, RANTES, or MCP-1, (data not shown). These biomarker profiles can be used to assess immune responses in patients receiving immunotherapy, and form a prognostic group to detect in vivo biologic activity of the TMV-based immunotherapy. Accordingly, IFN-γ, TNF-α, IL-2, IL-12, I-18, I-22, and IL-23 are demonstrated biomarkers.

These cytokines can be used to evaluate anti-viral and anti-tumor cell-mediated immune responses. The co-administration of anti-CTLA-4 mAb and TMVs augmented immunotherapy-induced tumor-specific immune responses, as shown by IFN-γ expression (FIG. 5A-B).

### Example 3. Testing of the Modified TMV Immunotherapy

### Immunotherapy Testing

TNBC-1.

Tissue processing (TMV production) was performed in single batches, one per patient, using frozen tumors. Frozen tumors were minced and mechanically homogenized, then centrifuged and clarified. The recovered clarified portions were ultracentrifuged in a sucrose gradient to isolate the membrane vesicle fraction, then the interface layer is centrifuged to pellet TMVs. TMVs were then washed and stored in saline at −80° C. All product-contact equipment and supplies in TMV processing were single-use. TMVs were uniform in size (400-600 nm) (data not shown), for both 4T1 murine TMVs and TMVs prepared from human breast cancer tumor tissue. The TMV yield was also comparable for both mouse and human tumors (data not shown).

In the TMV modification process, the GPI-proteins were incorporated into the TMVs through protein transfer. Incorporation of ISMs was readily achieved and controlled by adjusting incubation time, temperature, and concentration of GPI-ISMs. The level of GPI-ISM incorporation were quantified by either FACS or ELISA, as done for TMV-GPI-hB7.1/hIL-12 (data not shown). TMVs were modified with a combination of IL12 and B7-1 without adversely affecting the incorporation or function of either molecule. Briefly, TMVs (e.g., 100 μg/mL) were added to GPI-ISMs and incubated at 37° C. for about 4 hours. The mixture was centrifuged and washed twice, then stored in saline at −80° C. Modified TMVs were then thawed out for immunotherapy when needed.

Characterization.

Characterization of the products is performed during process development studies. The GPI-Protein processes are scaled up to accommodate the volumes required for manufacturing of clinical material, and the TMV process is performed repeatedly using donor breast cancer tissue at full scale. The analytical methods for in-process and release testing have been developed. Potential Critical Quality Attributes for TNBC-1 are as follows: average particle diameter of 200-800 nm and incorporation of >50× background negative control for both B7-1 and IL-12. These quality control attributes as well as additional ones (such as functional activity assays for B7-1 and IL-12) are refined as more human tumor samples are included for analysis.

### Cell-Based Reporter Systems for Release Testing

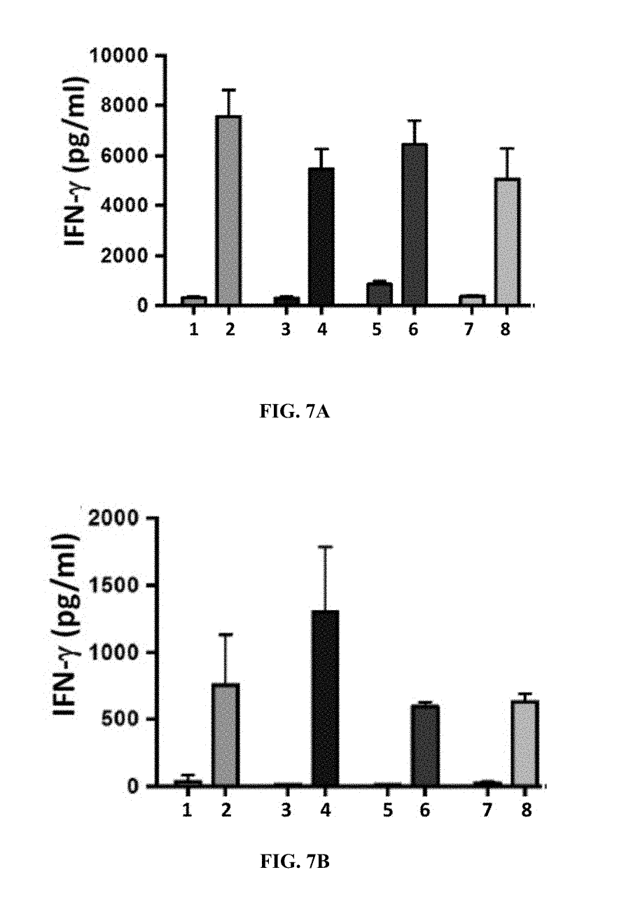

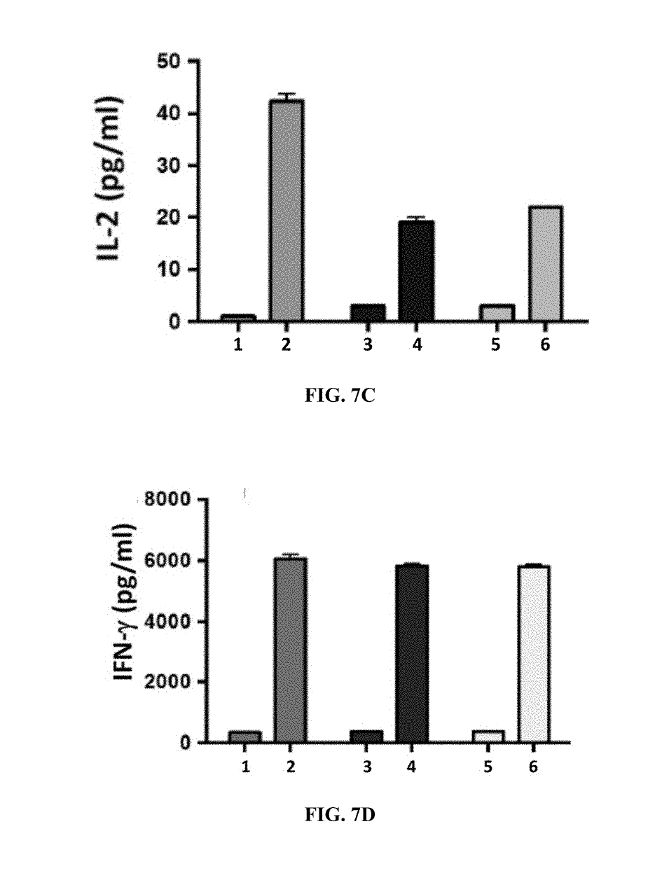

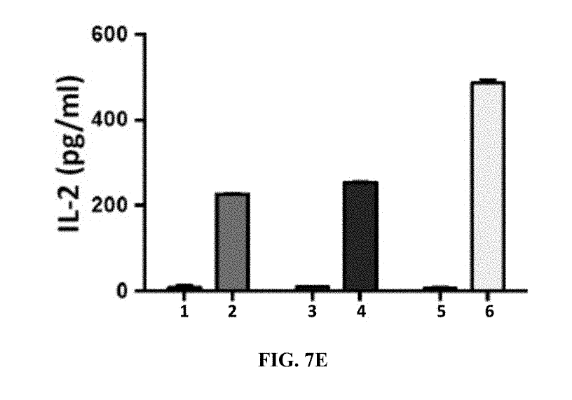

GPI-ISMs incorporated onto TMVs prepared from human TNBC cell lines and human breast cancer tissue retain functional activity. To test whether GPI-ISMs retained their activity in human TNBC TMVs, TMVs from four established human TNBC cell lines were prepared. GPI-ISMs incorporated into these TMVs stimulated activated human PBMC, Jurkat E6. 1, NK-92 MI or and NK-92 cells effectively compared to unincorporated TMVs (FIG. 7A-E). In addition, NK-92 or NK-92 MI cells can be used to detect both GPI-B7-1 and GPI-IL-12. This was accomplished by using blocking antibodies to either IL-12 (clone C8.6) or B7-1 (clone PSRM3) to measure respective contribution of each GPI-ISM. To confirm whether GPI-ISMs incorporated onto TMVs prepared from patient derived breast cancer tissue also stimulates immune cells, TMVs from three patient derived tumor samples were prepared and incorporated with GPI-ISMs. The TMV yield was consistent among the three tissues and effectively stimulated IL-2 and IFN-gamma secretion by Jurkat E6.1 and NK-92 cells, respectively, in the presence of anti-CD3 or PMA (FIG. 7C, 7E). These biological activity assays are useful during the preparation and release of a clinical product. In addition, other reporter cells can be used for detection of either human or murine IL-12, such as HEK-Blue IL-12 cells from Invivogen. Refer to the brief description of FIG. 7(A-E) for specific uses of ISMs in the TMVs.

Description of HEK Blue Cells: IL-12 Sensor Cells.

HEK-Blue™ IL-12 cells (InvivoGen Inc, California) are designed to detect bioactive human and mouse IL-12 by monitoring the activation of the STAT-4 pathway. These cells were generated by stably introducing the human genes for the IL-12 receptor and the genes of the IL-12 signaling pathway into HEK293 cells. Furthermore, these cells express a STAT4-inducible SEAP reporter gene. Binding of mouse or human IL-12 to the IL-12R on the surface of HEK-BLUE™ IL-12 cells triggers a signaling cascade leading to the activation STAT-4 with the subsequent production of SEAP. Detection of SEAP in the supernatant of HEK-BLUE™ IL-12 cells can be readily assessed using QUANTI-BLUE™. The detection range is 1-100 ng/ml for human and mouse IL-12, which is within the limits needed to detect incorporated GPI-IL-12 in TMVs (approximately 50-60 ng/ml). QUANTI-BLUE™ is a colorimetric enzyme assay developed to determine alkaline phosphatase activity (AP) in a biological sample, such as supernatants of cell cultures. In particular, QUANTI-BLUE™ provides an easy and rapid means to detect and quantify secreted embryonic alkaline phosphatase (SEAP), a reporter widely used for in vitro and in vivo analytical studies.

In the presence of alkaline phosphatase, the color of QUANTI-BLUE™ changes from pink to purple/blue. The intensity of the blue hue reflects the activity of AP. The levels of AP can be determined qualitatively with the naked eye or quantitatively using a spectrophotometer at 620-655 nm. QUANTI-BLUE™ is useful to monitor activation in reporter cell lines. HEK-BLUE™ IL-12 cells are used to validate the functionality of recombinant native or engineered human or mouse IL-12.

### Example 4: HER-2+ Breast Cancer Cells Expressing GPI-Anchored Cytokines Induce Long Lasting Antitumor Memory Response

D2F2 is a murine breast cancer cell line which can be transfected with human hHER-2 (the transfected cell line hereinafter referred to as D2F2/E2). Female BALB/c mice were challenged with D2F2/E2 cells or D2F2/E2 cells further transfected with GPI-IL-12 or GPI-GM-CSF (wherein GM-CSF refers to granulocyte macrophage colony stimulating factor). While the wild-type challenged mice developed tumors, the mice challenged with transfected D2F2/E2 cells were protected. Protected mice were re-challenged with D2F2/E2 cells 3 months later and D2F2 cells 4 months later. All the mice challenged with D2F2 or D2F2/E2 were protected. Strong antibody response against HER-2 and D2F2 cells were observed in these mice. Results showed that long lasting protective anti-tumor memory response against D2F2 and D2F2/E2 was generated by vaccination with D2F2/E2 cells expressing GPI-anchored immunostimulatory agents.

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

What is claimed is:

1. A method for treating a subject having, or at risk of having, a triple negative breast cancer, comprising administering to the subject a therapeutically effective amount of
a. a tumor membrane vesicle (TMV), wherein the TMV comprises a lipid membrane, and a B7-1 and/or IL-12 molecule anchored to the lipid membrane, and
b. an immunotherapeutic agent.

2. The method of claim 1, wherein the TMV comprises a B7-1 and a IL-12 molecule anchored to the lipid membrane.

3. The method of claim 1, wherein the TMV further comprises an antigen molecule anchored to the lipid membrane.

4. The method of claim 1, wherein the triple negative breast cancer is a metastatic triple negative breast cancer.

5. The method of claim 1, wherein the immunotherapeutic agent comprises one or more of an anti-CTLA4 antibody, an anti-PD1 antibody, and an anti-PD-L1 antibody.

6. The method of claim 1, wherein the immunotherapeutic agent is an anti-CTLA-4 antibody.

7. The method of claim 1, wherein the treatment reduces metastasis of the triple negative breast cancer.

8. The method of claim 1, wherein the treatment reduces the size of a tumor.

9. The method of claim 1, wherein the subject is a human.

10. A method for predicting the likelihood a subject having a cancer will respond therapeutically to a therapy administered to the subject, the therapy comprising administering a therapeutically effective amount of an immunotherapeutic agent and a tumor membrane vesicle (TMV), wherein the TMV comprises a lipid membrane, and a B7-1 and/or IL-12 molecule anchored to the lipid membrane, wherein the method for predicting comprises:
a. obtaining a blood or serum sample from the subject;
b. measuring protein expression levels of biomarkers in the sample,
wherein the biomarkers include at least IFN-gamma, TNF-alpha, and IL-2, and
wherein an increase in the levels of the biomarkers as compared to a control indicates an increased likelihood the subject will respond therapeutically to the therapy; and
c. advising the subject of the increased likelihood the subject will respond therapeutically to the therapy when the relative levels of the biomarkers increase or advising the subject of the decreased likelihood the subject will respond therapeutically to the therapy when the relative levels of the biomarkers do not increase.

11. The method of claim 10, wherein the subject is advised of the increased likelihood the subject will respond therapeutically to the therapy, further comprising treating the subject with an effective amount of the therapy.

12. The method of claim 10, wherein the TMV further comprises an antigen molecule anchored to the lipid membrane.

13. The method of claim 12, wherein the antigen molecule is selected from HER-2, PSA, and PAP.

14. The method of claim 12, wherein the antigen molecule is HER-2.

15. The method of claim 10, wherein the therapy further comprises an adjuvant.

16. The method of claim 15, wherein the TMV further comprises GM-CSF anchored to the lipid membrane.

17. The method of claim 10, wherein the cancer is breast cancer.

18. The method of claim 10, wherein the cancer is triple-negative breast cancer.

19. The method of claim 10, wherein the biomarkers include at least IFN-gamma, TNF-alpha, IL-2, and IL-12.

20. The method of claim 10, wherein the biomarkers include at least IFN-gamma, TNF-alpha, IL-2, and IL-18.

21. The method of claim 10, wherein the biomarkers include at least IFN-gamma, TNF-alpha, IL-2, IL-12 and IL-18.

22. The method of claim 10, wherein the biomarkers include at least IFN-gamma, TNF-alpha, IL-2, IL-12, IL-18, IL-22, and IL-23.

23. The method of claim 10, wherein the immunotherapeutic agent comprises one or more of an anti-CTLA4 antibody, an anti-PD1 antibody, and an anti-PD-L1 antibody.

24. The method of claim 10, wherein the immunotherapeutic agent is an anti-CTLA4 antibody.

25. A method of testing a tumor membrane vesicle (TMV) composition comprising:
a. combining the TMV composition with one or more human reporter cells in a sample; and
b. determining the amount of IL-2, IFN-gamma, or other reporter molecule in the sample after the combination;
c. wherein the TMV comprises a lipid membrane, and a B7-1 and/or IL-12 molecule anchored to the lipid membrane; and
d. wherein an increase in the amount of IL-2, IFN-gamma or other reporter molecule in the sample indicates an active TMV composition.
